(19) United States
(12) Patent Application Publication
(10) Pub. No.: US 2011/0183345 A1
(43) Pub. Date: Jul. 28, 2011

(54) COMPOSITIONS FOR USE IN IDENTIFICATION OF STREPTOCOCCUS PNEUMONIAE

(75) Inventors: Christian Massire, Carlsbad, CA (US); Rachael Kreft, Pittsburgh, PA (US); Lawrence B. Blyn, Mission Viejo, CA (US); David J. Ecker, Encinitas, CA (US); Feng Li, San Diego, CA (US); Rangarajan Sampath, San Diego, CA (US)

(73) Assignee: Ibis Biosciences, Inc., Carlsbad, CA (US)

(21) Appl. No.: 13/122,373

(22) PCT Filed: Sep. 30, 2009
(86) PCT No.: PCT/US09/59055

§ 371 Date: Apr. 1, 2011

Related U.S. Application Data
(60) Provisional application No. 61/102,715, filed on Oct. 3, 2008.

(51) Int. Cl. C12Q 1/68; H01J 49/26

(52) U.S. Cl. 435/6.12; 435/6.15; 250/281

(57) ABSTRACT

The present invention relates generally to the identification of Streptococcus pneumoniae, such as antibiotic resistant Streptococcus pneumoniae, and provides methods, compositions, kits and systems useful for this purpose when combined, for example, with molecular mass or base composition analysis.

### CROSS REFERENCE TO RELATED APPLICATIONS

The present application claims priority to U.S. Provisional Application No. 61/102,715, filed Oct. 3, 2008, which is incorporated by reference in its entirety.

### FIELD OF THE INVENTION

The present invention relates generally to the identification of Streptococcus pneumoniae, such as antibiotic resistant Streptococcus pneumoniae, and provides methods, compositions, kits and systems useful for this purpose when combined, for example, with molecular mass or base composition analysis.

### BACKGROUND OF THE INVENTION

Streptococcus pneumoniae, a member of the genus Streptococcus, is a Gram-positive, alpha-hemolytic diplococcus that causes many types of infectious disease including, for example, pneumonia, sinusitis, otitis media, meningitis, osteomyelitis, septic arthritis, sinusitis, pharyngitis, endocarditis, pericarditis, septicemia and bacteremia, peritonitis, cellulitis and tissue abscess. Of over 90 different serotypes of Streptococcus pneumoniae, some cause disease more commonly than others. Penicillin resistance of Streptococcus pneumoniae is increasing, as is resistance to other antibiotics including, for example, resistance to cephalosporins, macrolides (e.g., erythromycin), tetracycline, clindamycin, quinolones (e.g., levofloxacin and moxifloxacin), trimethoprim/sulfinethoxazole, and vancomycin.

The emergence of antibiotic-resistant Streptococcus pneumoniae is a serious infection control issue in hospitals and public health settings. Active screening for antibiotic-resistant Streptococcus pneumoniae in clinical specimens is recommended to limit the spread of antimicrobial resistance in high-risk patients. Accurate identification of the Streptococcus pneumoniae serogroups and serotypes, along with detection of one or more markers of antibiotic resistance, is critical for control and management of Streptococcus pneumoniae.

### SUMMARY OF THE INVENTION

The present invention relates generally to the detection and identification of Streptococcus pneumoniae (e.g., antibiotic resistant Streptococcus pneumoniae), and provides methods, compositions, systems and kits useful for this purpose when combined, for example, with molecular mass or base composition analysis. The compositions and methods described herein find use in a variety of biological sample analysis techniques, and are not limited to processes that employ or require molecular mass or base composition analysis. For example, primers described herein find use in a variety of research, surveillance, and diagnostic approaches that utilize one or more primers, including a variety of approaches that employ the polymerase chain reaction.

To further illustrate, in certain embodiments the invention provides for the rapid detection and characterization of Streptococcus pneumoniae. The primer pairs described herein, for example, may be used to detect members of Streptococcus pneumoniae serotypes, to determine the presence or absence of pbp2x, parC, gyrAiii, pbp2b, ermB, pbp1a, and mefE genoytpes, and to determine an antibiotic resistance profile. In addition to compositions and kits that include one or more of the primer pairs described herein, the invention also provides related methods and systems.

In one aspect, the present invention provides a composition comprising at least one purified oligonucleotide primer pair that comprises forward and reverse primers, wherein said primer pair comprises nucleic acid sequences that are substantially complementary to nucleic acid sequences of two or more different bioagents belonging to the Streptococcus pneumoniae serotypes, wherein the primer pair is configured to produce amplicons comprising different base compositions that correspond to the two or more different bioagents.

In some embodiments, the present invention provides compositions comprising at least one purified oligonucleotide primer pair that comprises forward and reverse primers about 15 to 35 nucleobases in length, wherein the forward primer comprises at least 70% identity (e.g., 70% . . . 75% . . . 90% . . . 95% . . . 100%) with a sequence selected from SEQ ID NOS: 1-40, and 81-93, and wherein the reverse primer comprises at least 70% identity (e.g., 70% . . . 75% . . . 90% . . . 95% . . . 100%) with a sequence selected from SEQ ID NOS: 41-80 and 93-104. Typically, the primer pair is configured to hybridize with Streptococcus pneumoniae (e.g., antibiotic resistant Streptococcus pneumoniae) nucleic acids. In further embodiments, the primer pair is selected from the group of primer pair sequences consisting of: SEQ ID NOS: 1:41, 2:42, 3:43, 4:44, 5:45, 6:46, 7:47, 8:48, 9:49, 10:50, 11:51, 12:52, 13:53, 14:54, 15:55, 16:56, 17:57, 18:58, 19:59, 20:60, 21:61, 22:62, 23:63, 24:64, 25:65, 26:66, 27:67, 28:68, 29:69, 30:70, 31:71, 32:72, 33:73, 34:74, 35:75, 36:76, 37:77, 38:78, 39:79, and 40:80. In other embodiments, the primer pair is selected from the group of primer pair sequences consisting of: SEQ ID NOS: 81:93, 82:94, 83:95, 84:96, 85:97, 86:98, 87:99, 88:100, 89:101, 90:102, 91:103, and 92:104. In some embodiments, the primer pair is specific for detection of Streptococcus pneumoniae wciN, wchA and/or wciO gene regions. In certain embodiments, the forward and/or reverse primer has a base length selected from the group consisting of: 15, 16, 17, 18, 19, 20, 21, 22, 23, 24, 25, 26, 27, 28, 29, 30, 31, 32, 33, 34, or 34 nucleotides, although both shorter and longer primers may be used.

In another aspect, the invention provides a purified oligonucleotide primer pair, comprising a forward primer and a reverse primer that each independently comprises 14 to 40 consecutive nucleobases selected from the primer pair sequences shown in Table 1 and/or Table 2, which primer pair is configured to generate an amplicon between about 50 and 150 consecutive nucleobases in length.

In another aspect, the invention provides a kit comprising at least one purified oligonucleotide primer pair that comprises forward and reverse primers that are about 20 to 35 nucleobases in length, and wherein the forward primer comprises at least 70%, at least 80%, at least 90%, at least 95%, or at least 100% sequence identity with a sequence selected from the group consisting of SEQ ID NOS: 1-40 and 81-92, and the reverse primer comprises at least 70% sequence identity (e.g., 75%, 85%, or 95%) with a sequence selected from the group consisting of SEQ ID NOS: 41-80 and 93-104. In some embodiments, the kit comprises a primer pair that is a broad range survey primer pair (e.g., specific for nucleic acid of a housekeeping gene found in many or all members of a category of organism, such as ribosomal RNA encoding genes in bacteria).

In other embodiments, the amplicons produced with the primers are 45 to 200 nucleobases in length (e.g., 45 . . . 75 . . . 125 . . . 175 . . . 200). In some embodiments, a non-templated T residue on the 5′-end of said forward and/or reverse primer is removed. In still other embodiments, the forward and/or reverse primer further comprises a non-templated T residue on the 5′-end. In additional embodiments, the forward and/or reverse primer comprises at least one molecular mass modifying tag. In some embodiments, the forward and/or reverse primer comprises at least one modified nucleobase. In further embodiments, the modified nucleobase is 5-propynyluracil or 5-propynylcytosine. In other embodiments, the modified nucleobase is a mass modified nucleobase. In still other embodiments, the mass modified nucleobase is 5-Iodo-C. In additional embodiments, the modified nucleobase is a universal nucleobase. In some embodiments, the universal nucleobase is inosine. In certain embodiments, kits comprise the compositions described herein.

In particular embodiments, the present invention provides methods of determining a presence of Streptococcus pneumoniae in at least one sample, the method comprising: (a) amplifying one or more (e.g., two or more, three or more, four or more, etc.; one to two, one to three, one to four, etc.; two, three, four, etc.) segments of at least one nucleic acid from the sample using at least one purified oligonucleotide primer pair that comprises forward and reverse primers that are about 20 to 35 nucleobases in length, and wherein the forward primer comprises at least 70% (e.g., 70% . . . 75% . . . 90% . . . 95% . . . 100%) sequence identity with a sequence selected from the group consisting of SEQ ID NOS: 1-40 and 81-92, and the reverse primer comprises at least 70% (e.g., 70% . . . 75% . . . 90% . . . 95% . . . 100%) sequence identity with a sequence selected from the group consisting of SEQ ID NOS: 41-80 and 93-104 to produce at least one amplification product; and (b) detecting the amplification product, thereby determining the presence of Streptococcus pneumoniae in the sample.

In certain embodiments, step (b) comprises determining an amount of (i.e. quantifying) Streptococcus pneumoniae in the sample. In further embodiments, step (b) comprises detecting a molecular mass of the amplification product. In other embodiments, step (b) comprises determining a base composition of the amplification product, wherein the base composition identifies the number of A residues, C residues, T residues, G residues, U residues, analogs thereof and/or mass tag residues thereof in the amplification product, whereby the base composition indicates the presence of Streptococcus pneumoniae in the sample or identifies the pathogenicity of Streptococcus pneumoniae in the sample. In particular embodiments, the methods further comprise comparing the base composition of the amplification product to calculated or measured base compositions of amplification products of one or more known Streptococcus pneumoniae present in a database, for example, with the proviso that sequencing of the amplification product is not used to indicate the presence of or to identify Streptococcus pneumoniae, wherein a match between the determined base composition and the calculated or measured base composition in the database indicates the presence of or identifies the Streptococcus pneumoniae. In some embodiments, the identification of Streptococcus pneumoniae is at the genus levels, species level, serogroup level, serotype level, genotype level, or individual identity level.

In some embodiments, the present invention provides methods of identifying one or more Streptococcus pneumoniae bioagents in a sample, the method comprising: amplifying two or more segments of a nucleic acid from the one or more Streptococcus pneumoniae bioagents in the sample with two or more oligonucleotide primer pairs to obtain two or more amplification products (e.g., from a single bioagent); (b) determining two or more molecular masses and/or base compositions of the two or more amplification products; and (c) comparing the two or more molecular masses and/or the base compositions of the two or more amplification products with known molecular masses and/or known base compositions of amplification products of known Streptococcus pneumoniae bioagents produced with the two or more primer pairs to identify the one or more Streptococcus pneumoniae bioagents in the sample. In certain embodiments, the methods comprise identifying the one or more Streptococcus pneumoniae bioagents in the sample using three, four, five, six, seven, eight or more primer pairs. In other embodiments, the one or more Streptococcus pneumoniae bioagents in the sample cannot be identified using a single primer pair of the two or more primer pairs. In particular embodiments, the methods comprise obtaining the two or more molecular masses of the two or more amplification products via mass spectrometry. In certain embodiments, the methods comprise calculating the two or more base compositions from the two or more molecular masses of the two or more amplification products.

In some embodiments, the present invention provides methods of identifying one or more serotypes of Streptococcus pneumoniae in a sample, the method comprising: (a) amplifying two or more segments of a nucleic acid from the one or more Streptococcus pneumoniae in the sample with first and second oligonucleotide primer pairs to obtain two or more amplification products, wherein the first primer pair an amplicon that reveals species, and wherein the second primer pair produces an amplicon that reveals sub-species, serotype, strain, genotype-specific, or antibiotic resistance information; (b) determining two or more molecular masses and/or base compositions of the two or more amplification products; and (c) comparing the two or more molecular masses and/or the base compositions of the two or more amplification products with known molecular masses and/or known base compositions of amplification products of known Streptococcus pneumoniae produced with the first and second primer pairs to identify the Streptococcus pneumoniae in the sample. In some embodiments, the second primer pair amplifies a portion of a gene including, but not limited to pbp2x, parC, gyrA, pbp2b, ermB, pbp1a, and mefE.

In certain embodiments, the second primer pair comprises forward and reverse primers that are about 20 to 35 nucleobases in length, and wherein the forward primer comprises at least 70% sequence identity with a sequence selected from the group consisting of SEQ ID NOS: 1-40 and 81-92, and the reverse primer comprises at least 70% sequence identity with a sequence selected from the group consisting of SEQ ID NOS: 41-80 and 93-104 to produce at least one amplification product. In further embodiments, the obtaining the two or more molecular masses of the two or more amplification products is via mass spectrometry. In some embodiments, the methods comprise calculating the two or more base compositions from the two or more molecular masses of the two or more amplification products.

In some embodiments, the second primer pair is selected from the group of primer pair sequences consisting of: SEQ ID NOS: 1:41, 2:42, 3:43, 4:44, 5:45, 6:46, 7:47, 8:48, 9:49, 10:50, 11:51, 12:52, 13:53, 14:54, 15:55, 16:56, 17:57, 18:58, 19:59, 20:60, 21:61, 22:62, 23:63, 24:64, 25:65, 26:66, 27:67, 28:68, 29:69, 30:70, 31:71, 32:72, 33:73, 34:74, 35:75, 36:76, 37:77, 38:78, 39:79, and 40:80. In other embodiments, the second primer pair is selected from the group of primer pair sequences consisting of: SEQ ID NOS: 81:93, 82:94, 83:95, 84:96, 85:97, 86:98, 87:99, 88:100, 89:101, 90:102, 91:103, and 92:104. In further embodiments, the determining the two or more molecular masses and/or base compositions is conducted without sequencing the two or more amplification products. In certain embodiments, Streptococcus pneumoniae in the sample cannot be identified using a single primer pair of the first and second primer pairs. In other embodiments, the Streptococcus pneumoniae in the sample is identified by comparing three or more molecular masses and/or base compositions of three or more amplification products with a database of known molecular masses and/or known base compositions of amplification products of known Streptococcus pneumoniae produced with the first and second primer pairs, and a third primer pair.

In further embodiments, members of the first and second primer pairs hybridize to conserved regions of the nucleic acid that flank a variable region. In some embodiments, the variable region varies between at least two serotypes of Streptococcus pneumoniae. In particular embodiments, the variable region uniquely varies between at least two (e.g., 3, 4, 5, 6, 7, 8, 9, 10, . . . , 20, etc.) species, seroytpes, or genotypes of Streptococcus pneumoniae. In particular embodiments, the variable region uniquely varies between at least two types of antibiotic resistance genes.

In some embodiments, the present invention provides systems comprising: (a) a mass spectrometer configured to detect one or more molecular masses of amplicons produced using at least one purified oligonucleotide primer pair that comprises forward and reverse primers about 15 to 35 nucleobases in length, wherein the forward primer comprises at least 70% (e.g., 70% . . . 75% . . . 90% . . . 95% . . . 100%) identity with a sequence selected from SEQ ID NOs: 1-40 and 81-92, and wherein the reverse primer comprises at least 70% (e.g., 70% . . . 75% . . . 90% . . . 95% . . . 100%) identity with a sequence selected from SEQ ID NOS: 41-80 and 93-104; and (b) a controller operably connected to the mass spectrometer, the controller configured to correlate the molecular masses of the amplicons with one or more strains of Streptococcus pneumoniae identities. In certain embodiments, the second primer pair is selected from the group of primer pair sequences consisting of: SEQ ID NOS: 1:41, 2:42, 3:43, 4:44, 5:45, 6:46, 7:47, 8:48, 9:49, 10:50, 11:51, 12:52, 13:53, 14:54, 15:55, 16:56, 17:57, 18:58, 19:59, 20:60, 21:61, 22:62, 23:63, 24:64, 25:65, 26:66, 27:67, 28:68, 29:69, 30:70, 31:71, 32:72, 33:73, 34:74, 35:75, 36:76, 37:77, 38:78, 39:79, and 40:80. In other embodiments, the second primer pair is selected from the group of primer pair sequences consisting of: SEQ ID NOS: 81:93, 82:94, 83:95, 84:96, 85:97, 86:98, 87:99, 88:100, 89:101, 90:102, 91:103, and 92:104. In further embodiments, the controller is configured to determine base compositions of the amplicons from the molecular masses of the amplicons, which base compositions correspond to the one or more strain of Streptococcus pneumoniae. In particular embodiments, the controller comprises or is operably connected to a database of known molecular masses and/or known base compositions of amplicons of known species of Streptococcus pneumoniae produced with the primer pair.

In certain embodiments, the database comprises molecular mass information for at least three different bioagents. In other embodiments, the database comprises molecular mass information for at least 2 . . . 10 . . . 50 . . . 100 . . . 1000 . . . 10,000, or 100,000 different bioagents. In particular embodiments, the molecular mass information comprises base composition data. In some embodiments, the base composition data comprises at least 10 . . . 50 . . . 100 . . . 500 . . . 1000 . . . 1000 . . . 10,000 . . . or 100,000 unique base compositions. In other embodiments, the database comprises molecular mass information for a bioagent from two or more serotypes selected from the species Streptococcus pneumoniae. In some embodiments, the database comprises molecular mass information for a bioagent from each of the Streptococci. In further embodiments, the database comprises molecular mass information for an Streptococcus pneumoniae bioagent. In further embodiments, the database is stored on a local computer. In particular embodiments, the database is accessed from a remote computer over a network. In further embodiments, the molecular mass in the database is associated with bioagent identity. In certain embodiments, the molecular mass in the database is associated with bioagent geographic origin. In particular embodiments, bioagent identification comprises interrogation of the database with two or more different molecular masses (e.g., 2, 3, 4, 5, . . . 10 . . . 25 or more molecular masses) associated with the bioagent.

In some embodiments, the present invention provides a method of detecting an infection with two or more bioagents in a subject comprising providing a sample from the subject, amplifying two or more segments of a nucleic acid from one or more Streptococcus pneumoniae bioagents in the sample with two or more oligonucleotide primer pairs to obtain two or more amplification products, determining two or more molecular masses and/or base compositions of the two or more amplification products, and comparing the two or more molecular masses and/or the base compositions of the two or more amplification products with known molecular masses and/or known base compositions of amplification products of known Streptococcus pneumoniae bioagents produced with the two or more primer pairs to identify the two or more Streptococcus pneumoniae bioagents. In some embodiments, the subject is a patient undergoing critical care or intensive care.

### BRIEF DESCRIPTION OF THE DRAWINGS

The foregoing summary and detailed description is better understood when read in conjunction with the accompanying drawings which are included by way of example and not by way of limitation.

FIG. 1 shows a process diagram illustrating one embodiment of the primer pair selection process.

FIG. 2 shows a process diagram illustrating one embodiment of the primer pair validation process. Here select primers are shown meeting test criteria. Criteria include but are not limited to, the ability to amplify targeted Streptococcus pneumoniae nucleic acid, the ability to exclude non-target bioagents, the ability to not produce unexpected amplicons, the ability to not dimerize, the ability to have analytical limits of detection of ≦100 genomic copies/reaction, and the ability to differentiate amongst different target organisms.

FIG. 3 shows a process diagram illustrating an embodiment of the calibration method.

FIG. 4 shows a block diagram showing a representative system.

### DETAILED DESCRIPTION OF EMBODIMENTS

It is to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this invention pertains. In describing and claiming the present invention, the following terminology and grammatical variants will be used in accordance with the definitions set forth below.

As used herein, the term “about” means encompassing plus or minus 10%. For example, about 200 nucleotides refers to a range encompassing between 180 and 220 nucleotides.

As used herein, the term “amplicon” or “bioagent identifying amplicon” refers to a nucleic acid generated using the primer pairs described herein. The amplicon is typically double stranded DNA; however, it may be RNA and/or DNA:RNA. In some embodiments, the amplicon comprises DNA complementary to Streptococcus pneumoniae (e.g., antibiotic resistant Streptococcus pneumoniae) RNA, DNA, or cDNA. In some embodiments, the amplicon comprises sequences of conserved regions/primer pairs and intervening variable region. As discussed herein, primer pairs are configured to generate amplicons from Streptococcus pneumoniae nucleic acid (e.g., antibiotic resistant Streptococcus pneumoniae nucleic acid). As such, the base composition of any given amplicon may include the primer pair, the complement of the primer pair, the conserved regions and the variable region from the bioagent that was amplified to generate the amplicon. One skilled in the art understands that the incorporation of the designed primer pair sequences into an amplicon may replace the native sequences at the primer binding site, and complement thereof. In certain embodiments, after amplification of the target region using the primers the resultant amplicons having the primer sequences are used to generate the molecular mass data. Generally, the amplicon further comprises a length that is compatible with mass spectrometry analysis. Bioagent identifying amplicons generate base compositions that are preferably unique to the identity of a bioagent (e.g., antibiotic resistant Streptococcus pneumoniae).

Amplicons typically comprise from about 45 to about 200 consecutive nucleobases (i.e., from about 45 to about 200 linked nucleosides). One of ordinary skill in the art will appreciate that this range expressly embodies compounds of 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, and 200 nucleobases in length. One of ordinary skill in the art will further appreciate that the above range is not an absolute limit to the length of an amplicon, but instead represents a preferred length range. Amplicon lengths falling outside of this range are also included herein so long as the amplicon is amenable to calculation of a base composition signature as herein described.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. Generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using reverse transcription (RT)-PCR is a form of amplification. Furthermore, the generation of multiple RNA molecules from a single DNA molecule during the process of transcription is also a form of amplification.

As used herein, “bacterial nucleic acid” includes, but is not limited to, DNA, RNA, or DNA that has been obtained from bacterial RNA, such as, for example, by performing a reverse transcription reaction. Bacterial RNA can either be single-stranded (of positive or negative polarity) or double-stranded.

As used herein, the term “base composition” refers to the number of each residue comprised in an amplicon or other nucleic acid, without consideration for the linear arrangement of these residues in the strand(s) of the amplicon. The amplicon residues comprise, adenosine (A), guanosine (G), cytidine, (C), (deoxy)thymidine (T), uracil (U), inosine (I), nitroindoles such as 5-nitroindole or 3-nitropyrrole, dP or dK (Hill F et al., Polymerase recognition of synthetic oligodeoxyribonucleotides incorporating degenerate pyrimidine and purine bases. Proc Natl Acad Sci USA. 1998 Apr. 14; 95(8):4258-63), an acyclic nucleoside analog containing 5-nitroindazole (Van Aerschot et al., Nucleosides and Nucleotides, 1995, 14, 1053-1056), the purine analog 1-(2-deoxy-beta-D-ribofuranosyl)-imidazole-4-carboxamide, 2,6-diaminopurine, 5-propynyluracil, 5-propynylcytosine, phenoxazines, including G-clamp, 5-propynyl deoxy-cytidine, deoxy-thymidine nucleotides, 5-propynylcytidine, 5-propynyluridine and mass tag modified versions thereof, including 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine-5′-triphosphate, 5-iodo-2′-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate, O6-methyl-2′-deoxyguanosine-5′-triphosphate, N2-methyl-2′-deoxyguanosine-5′-triphosphate, 8-oxo-2′-deoxyguanosine-5′-triphosphate or thiothymidine-5′-triphosphate. In some embodiments, the mass-modified nucleobase comprises ¹⁵N or ¹³C or both ¹⁵N and ¹³C. In some embodiments, the non-natural nucleosides used herein include 5-propynyluracil, 5-propynylcytosine and inosine. Herein the base composition for an unmodified DNA amplicon is notated as A_{w}GₓC_{y}T_{z}, wherein w, x, y and z are each independently a whole number representing the number of said nucleoside residues in an amplicon. Base compositions for amplicons comprising modified nucleosides are similarly notated to indicate the number of said natural and modified nucleosides in an amplicon. Base compositions are calculated from a molecular mass measurement of an amplicon, as described below. The calculated base composition for any given amplicon is then compared to a database of base compositions. A match between the calculated base composition and a single database entry reveals the identity of the bioagent.

As used herein, a “base composition probability cloud” is a representation of the diversity in base composition resulting from a variation in sequence that occurs among different isolates of a given species, family or genus. Base composition calculations for a plurality of amplicons are mapped on a pseudo four-dimensional plot. Related members in a family, genus or species typically cluster within this plot, forming a base composition probability cloud.

As used herein, the term “base composition signature” refers to the base composition generated by any one particular amplicon.

As used herein, a “bioagent” means any biological organism or component thereof or a sample containing a biological organism or component thereof, including microorganisms or infectious substances, or any naturally occurring, bioengineered or synthesized component of any such microorganism or infectious substance or any nucleic acid derived from any such microorganism or infectious substance. Those of ordinary skill in the art will understand fully what is meant by the term bioagent given the instant disclosure. Still, a non-exhaustive list of bioagents includes: cells, cell lines, human clinical samples, mammalian blood samples, cell cultures, bacterial cells, viruses, viroids, fungi, protists, parasites, rickettsiae, protozoa, animals, mammals or humans. Samples may be alive, non-replicating or dead or in a vegetative state (for example, vegetative bacteria or spores). Preferably, the bioagent is a Streptococcus pneumoniae, such as, for example, antibiotic resistant Streptococcus pneumoniae.

As used herein, a “bioagent division” is defined as group of bioagents above the species level and includes but is not limited to, orders, families, genus, classes, clades, genera or other such groupings of bioagents above the species level.

As used herein, “broad range survey primers” are primers designed to identify an unknown bioagent as a member of a particular biological division (e.g., an order, family, class, clade, or genus). However, in some cases the broad range survey primers are also able to identify unknown bioagents at the species or sub-species level. As used herein, “division-wide primers” are primers designed to identify a bioagent at the species level and “drill-down” primers are primers designed to identify a bioagent at the sub-species level. As used herein, the “sub-species” level of identification includes, but is not limited to, strains, subtypes, serogroups, serovars, serotypes, variants, and isolates. Drill-down primers are not always required for identification at the sub-species level because broad range survey intelligent primers may, in some cases provide sufficient identification resolution to accomplishing this identification objective.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon

The term “conserved region” in the context of nucleic acids refers to a nucleobase sequence (e.g., a subsequence of a nucleic acid, etc.) that is the same or similar in two or more different regions or segments of a given nucleic acid molecule (e.g., an intramolecular conserved region), or that is the same or similar in two or more different nucleic acid molecules (e.g., an intermolecular conserved region). To illustrate, a conserved region may be present in two or more different taxonomic ranks (e.g., two or more different genera, two or more different species, two or more different serotypes, and the like) or in two or more different nucleic acid molecules from the same organism. To further illustrate, in certain embodiments, nucleic acids comprising at least one conserved region typically have between about 70%-100%, between about 80-100%, between about 90-100%, between about 95-100%, or between about 99-100% sequence identity in that conserved region. A conserved region may also be selected or identified functionally as a region that permits generation of amplicons via primer extension through hybridization of a completely or partially complementary primer to the conserved region for each of the target sequences to which conserved region is conserved.

The term “correlates” refers to establishing a relationship between two or more things. In certain embodiments, for example, detected molecular masses of one or more amplicons indicate the presence or identity of a given bioagent in a sample. In some embodiments, base compositions are calculated or otherwise determined from the detected molecular masses of amplicons, which base compositions indicate the presence or identity of a given bioagent in a sample.

As used herein, in some embodiments the term “database” is used to refer to a collection of base composition molecular mass data. In other embodiments the term “database” is used to refer to a collection of base composition data. The base composition data in the database is indexed to bioagents and to primer pairs. The base composition data reported in the database comprises the number of each nucleoside in an amplicon that would be generated for each bioagent using each primer. The database can be populated by empirical data. In this aspect of populating the database, a bioagent is selected and a primer pair is used to generate an amplicon. The amplicon's molecular mass is determined using a mass spectrometer and the base composition calculated therefrom without sequencing i.e., without determining the linear sequence of nucleobases comprising the amplicon. Note that base composition entries in the database may be derived from sequencing data (i.e., known sequence information), but the base composition of the amplicon to be identified is determined without sequencing the amplicon. An entry in the database is made to correlate the base composition with the bioagent and the primer pair used. The database may also be populated using other databases comprising bioagent information. For example, using the GenBank database it is possible to perform electronic PCR using an electronic representation of a primer pair. This in silico method may provide the base composition for any or all selected bioagent(s) stored in the GenBank database. The information may then be used to populate the base composition database as described above. A base composition database can be in silico, a written table, a reference book, a spreadsheet or any form generally amenable to databases. Preferably, it is in silico on computer readable media.

The terms “detect”, “detecting” or “detection” refers to an act of determining the existence or presence of one or more targets (e.g., bioagent nucleic acids, amplicons, etc.) in a sample.

As used herein, the term “etiology” refers to the causes or origins, of diseases or abnormal physiological conditions.

As used herein, the term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length sequence or fragment thereof are retained.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to nucleic acid sequences that are not found naturally associated with the gene sequences in the chromosome or are associated with portions of the chromosome not found in nature (e.g., genes expressed in loci where the gene is not normally expressed).

The terms “homology,” “homologous” and “sequence identity” refer to a degree of identity. There may be partial homology or complete homology. A partially homologous sequence is one that is less than 100% identical to another sequence. Determination of sequence identity is described in the following example: a primer 20 nucleobases in length which is otherwise identical to another 20 nucleobase primer but having two non-identical residues has 18 of 20 identical residues (18/20=0.9 or 90% sequence identity). In another example, a primer 15 nucleobases in length having all residues identical to a 15 nucleobase segment of a primer 20 nucleobases in length would have 15/20=0.75 or 75% sequence identity with the 20 nucleobase primer. In context of the present invention, sequence identity is meant to be properly determined when the query sequence and the subject sequence are both described and aligned in the 5′ to 3′ direction. Sequence alignment algorithms such as BLAST, will return results in two different alignment orientations. In the Plus/Plus orientation, both the query sequence and the subject sequence are aligned in the 5′ to 3′ direction. On the other hand, in the Plus/Minus orientation, the query sequence is in the 5′ to 3′ direction while the subject sequence is in the 3′ to 5′ direction. It should be understood that with respect to the primers of the present invention, sequence identity is properly determined when the alignment is designated as Plus/Plus. Sequence identity may also encompass alternate or “modified” nucleobases that perform in a functionally similar manner to the regular nucleobases adenine, thymine, guanine and cytosine with respect to hybridization and primer extension in amplification reactions. In a non-limiting example, if the 5-propynyl pyrimidines propyne C and/or propyne T replace one or more C or T residues in one primer which is otherwise identical to another primer in sequence and length, the two primers will have 100% sequence identity with each other. In another non-limiting example, inosine (I) may be used as a replacement for G or T and effectively hybridize to C, A or U (uracil). Thus, if inosine replaces one or more C, A or U residues in one primer which is otherwise identical to another primer in sequence and length, the two primers will have 100% sequence identity with each other. Other such modified or universal bases may exist which would perform in a functionally similar manner for hybridization and amplification reactions and will be understood to fall within this definition of sequence identity.

As used herein, “housekeeping gene” or “core viral gene” refers to a gene encoding a protein or RNA involved in basic functions required for survival and reproduction of a bioagent. Housekeeping genes include, but are not limited to, genes encoding RNA or proteins involved in translation, replication, recombination and repair, transcription, nucleotide metabolism, amino acid metabolism, lipid metabolism, energy generation, uptake, secretion and the like.

As used herein, the term “hybridization” or “hybridize” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, the melting temperature (Tₘ) of the formed hybrid, and the G:C ratio within the nucleic acids. A single molecule that contains pairing of complementary nucleic acids within its structure is said to be “self-hybridized.” An extensive guide to nucleic hybridization may be found in Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993), which is incorporated by reference.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method.

As used herein, “intelligent primers” or “primers” or “primer pairs,” in some embodiments, are oligonucleotides that are designed to bind to conserved sequence regions of one or more bioagent nucleic acids to generate bioagent identifying amplicons. In some embodiments, the bound primers flank an intervening variable region between the conserved binding sequences. Upon amplification, the primer pairs yield amplicons e.g., amplification products that provide base composition variability between the two or more bioagents. The variability of the base compositions allows for the identification of one or more individual bioagents from, e.g., two or more bioagents based on the base composition distinctions. In some embodiments, the primer pairs are also configured to generate amplicons amenable to molecular mass analysis. Further, the sequences of the primer members of the primer pairs are not necessarily fully complementary to the conserved region of the reference bioagent. For example, in some embodiments, the sequences are designed to be “best fit” amongst a plurality of bioagents at these conserved binding sequences. Therefore, the primer members of the primer pairs have substantial complementarity with the conserved regions of the bioagents, including the reference bioagent.

In some embodiments of the invention, the oligonucleotide primer pairs described herein can be purified. As used herein, “purified oligonucleotide primer pair,” “purified primer pair,” or “purified” means an oligonucleotide primer pair that is chemically-synthesized to have a specific sequence and a specific number of linked nucleosides. This term is meant to explicitly exclude nucleotides that are generated at random to yield a mixture of several compounds of the same length each with randomly generated sequence. As used herein, the term “purified” or “to purify” refers to the removal of one or more components (e.g., contaminants) from a sample.

As used herein, the term “molecular mass” refers to the mass of a compound as determined using mass spectrometry, for example, ESI-MS. Herein, the compound is preferably a nucleic acid. In some embodiments, the nucleic acid is a double stranded nucleic acid (e.g., a double stranded DNA nucleic acid). In some embodiments, the nucleic acid is an amplicon. When the nucleic acid is double stranded the molecular mass is determined for both strands. In one embodiment, the strands may be separated before introduction into the mass spectrometer, or the strands may be separated by the mass spectrometer (for example, electro-spray ionization will separate the hybridized strands). The molecular mass of each strand is measured by the mass spectrometer.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxyl-methyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP). As is used herein, a nucleobase includes natural and modified residues, as described herein.

An “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth Enzymol. 68:90-99; the phosphodiester method of Brown et al. (1979) Meth Enzymol. 68:109-151; the diethylphosphoramidite method of Beaucage et al. (1981) Tetrahedron Lett. 22:1859-1862; the triester method of Matteucci et al. (1981) J Am Chem Soc 103:3185-3191; automated synthesis methods; or the solid support method of U.S. Pat. No. 4,458,066, entitled “PROCESS FOR PREPARING POLYNUCLEOTIDES,” issued Jul. 3, 1984 to Caruthers et al., or other methods known to those skilled in the art. All of these references are incorporated by reference.

As used herein a “sample” refers to anything capable of being analyzed by the methods provided herein. In some embodiments, the sample comprises or is suspected to comprise one or more nucleic acids capable of analysis by the methods. Preferably, the samples comprise nucleic acids (e.g., DNA, RNA, cDNAs, etc.) from one or more Streptococcus pneumoniae. Samples can include, for example, evidence from a crime scene, blood, blood stains, semen, semen stains, bone, teeth, hair saliva, urine, feces, fingernails, muscle tissue, cigarettes, stamps, envelopes, dandruff, fingerprints, personal items, sputum, bile, cerebrospinal fluid, bronchoalveolar lavage, middle ear fluid, a tissue sample, an abscess sample, a tissue cavity swab, and the like. In some embodiments, the samples are “mixture” samples, which comprise nucleic acids from more than one subject or individual. In some embodiments, the methods provided herein comprise purifying the sample or purifying the nucleic acid(s) from the sample. In some embodiments, the sample is purified nucleic acid.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

As is used herein, the term “single primer pair identification” means that one or more bioagents can be identified using a single primer pair. A base composition signature for an amplicon may singly identify one or more bioagents.

As used herein, a “sub-species characteristic” is a genetic characteristic that provides the means to distinguish two members of the same bioagent species. For example, one bacterial strain may be distinguished from another bacterial strain of the same species by possessing a genetic change (e.g., for example, a nucleotide deletion, addition or substitution) in one of the viral genes, such as the RNA-dependent RNA polymerase.

As used herein, in some embodiments the term “substantial complementarity” means that a primer member of a primer pair comprises between about 70%-100%, or between about 80-100%, or between about 90-100%, or between about 95-100%, or between about 99-100% complementarity with the conserved binding sequence of a nucleic acid from a given bioagent. Similarly, the primer pairs provided herein may comprise between about 70%-100%, or between about 80-100%, or between about 90-100%, or between about 95-100% identity, or between about 99-100% sequence identity with the primer pairs disclosed in Tables 1 and 2. These ranges of complementarity and identity are inclusive of all whole or partial numbers embraced within the recited range numbers. For example, and not limitation, 75.667%, 82%, 91.2435% and 97% complementarity or sequence identity are all numbers that fall within the above recited range of 70% to 100%, therefore forming a part of this description. In some embodiments, any oligonucleotide primer pair may have one or both primers with less than 70% sequence homology with a corresponding member of any of the primer pairs of Tables 1 and 2 if the primer pair has the capability of producing an amplification product corresponding to the desired Streptococcus pneumoniae (e.g., antibiotic resistant Streptococcus pneumoniae) identifying amplicon.

A “system” in the context of analytical instrumentation refers a group of objects and/or devices that form a network for performing a desired objective.

As used herein, “triangulation identification” means the use of more than one primer pair to generate a corresponding amplicon for identification of a bioagent. The more than one primer pair can be used in individual wells, or vessels or in a multiplex PCR assay wherein each well contains two or more primer pairs. For example, a single well may comprise one or more primer pairs for Streptococcus pneumoniae multilocus sequence typing (MLST), together with one or more primer pairs specific for detection and identification of one or more Streptococcus pneumoniae serotypes. In some embodiments, the testing format and platform (e.g., a 96-well, or 384-well microtiter plate) comprises two or more multiplex wells. Alternatively, PCR reactions may be carried out in single wells or vessels comprising a different primer pair in each well or vessel. Following amplification the amplicons are pooled into a single well or container which is then subjected to molecular mass analysis. The combination of pooled amplicons can be chosen such that the expected ranges of molecular masses of individual amplicons are not overlapping and thus will not complicate identification of signals. Triangulation is a process of elimination, wherein a first primer pair identifies that an unknown bioagent may be one of a group of bioagents. Subsequent primer pairs are used in triangulation identification to further refine the identity of the bioagent amongst the subset of possibilities generated with the earlier primer pair. Triangulation identification is complete when the identity of the bioagent is determined. The triangulation identification process may also be used to reduce false negative and false positive signals, and enable reconstruction of the origin of hybrid or otherwise engineered bioagents. For example, identification of the three part toxin genes typical of B. anthracis (Bowen et al., J Appl Microbiol, 1999, 87, 270-278) in the absence of the expected compositions from the B. anthracis genome would suggest a genetic engineering event.

As used herein, the term “unknown bioagent” can mean, for example: (i) a bioagent whose existence is not known (for example, the SARS coronavirus was unknown prior to April 2003) and/or (ii) a bioagent whose existence is known (such as the well known bacterial species Staphylococcus aureus for example) but which is not known to be in a sample to be analyzed. For example, if the method for identification of coronaviruses disclosed in commonly owned U.S. patent Ser. No. 10/829,826 (incorporated herein by reference in its entirety) was employed prior to April 2003 to identify the SARS coronavirus in a clinical sample, both meanings of “unknown” bioagent would be applicable since the SARS coronavirus was unknown to science prior to April, 2003 and since it was not known what bioagent (in this case a coronavirus) was present in the sample. On the other hand, if the method of U.S. patent Ser. No. 10/829,826 was employed subsequent to April 2003 to identify the SARS coronavirus in a clinical sample, the second meaning (ii) of “unknown” bioagent would apply because the SARS coronavirus became known to science subsequent to April 2003 because it was not known what bioagent was present in the sample.

As used herein, the term “variable region” is used to describe a region that falls between any one primer pair described herein. The region possesses distinct base compositions between at least two bioagents, such that at least one bioagent can be identified at, for example, the family, genus, species or sub-species, strain or serotype level. The degree of variability between the at least two bioagents need only be sufficient to allow for identification using mass spectrometry analysis, as described herein.

As used herein, a “wobble base” is a variation in a codon found at the third nucleotide position of a DNA triplet. Variations in conserved regions of sequence are often found at the third nucleotide position due to redundancy in the amino acid code.

Provided herein are methods, compositions, kits, and related systems for the detection and identification of Streptococcus pneumoniae bioagents using bioagent identifying amplicons. To further illustrate, the methods and other aspects of the invention may be used to detect any member of the Streptococcus pneumoniae genus and identify the species; to genotypically characterize Streptococcus pneumoniae (e.g., antibiotic resistant Streptococcus pneumoniae) according to, for example, CDC-designated USA serotypes, to determine the presence or absence of virulence factor genes, and/or to determine an antibiotic resistance profile. In some embodiments, primers are selected to hybridize to conserved sequence regions of nucleic acids derived from a bioagent and which flank variable sequence regions to yield a bioagent identifying amplicon which can be amplified and which is amenable to molecular mass determination. In some embodiments, the molecular mass is converted to a base composition, which indicates the number of each nucleotide in the amplicon. Systems employing software and hardware useful in converting molecular mass data into base composition information are available from, for example, Ibis Biosciences, Inc. (Carlsbad, Calif.), for example the Ibis T5000 Biosensor System, and are described in U.S. patent application Ser. No. 10/754,415, filed Jan. 9, 2004, incorporated by reference herein in its entirety. In some embodiments, the molecular mass or corresponding base composition of one or more different amplicons is queried against a database of molecular masses or base compositions indexed to bioagents and to the primer pair used to generate the amplicon. A match of the measured base composition to a database entry base composition associates the sample bioagent to an indexed bioagent in the database. Thus, the identity of the unknown bioagent is determined. No prior knowledge of the unknown bioagent is necessary to make the identification. In some instances, the measured base composition associates with more than one database entry base composition. Thus, a second/subsequent primer pair is generally used to generate an amplicon, and its measured base composition is similarly compared to the database to determine its identity in triangulation identification. Furthermore, the methods and other aspects of the invention can be applied to rapid parallel multiplex analyses, the results of which can be employed in a triangulation identification strategy. Thus, in some embodiments, the present invention provides rapid throughput and does not require nucleic acid sequencing or knowledge of the linear sequences of nucleobases of the amplified target sequence for bioagent detection and identification.

Particular embodiments of the mass-spectrum based detection methods are described in the following patents, patent applications and scientific publications, all of which are herein incorporated by reference as if fully set forth herein: U.S. Pat. Nos. 7,108,974; 7,217,510; 7,226,739; 7,255,992; 7,312,036; 7,339,051; U.S. patent publication numbers 2003/0027135; 2003/0167133; 2003/0167134; 2003/0175695; 2003/0175696; 2003/0175697; 2003/0187588; 2003/0187593; 2003/0190605; 2003/0225529; 2003/0228571; 2004/0110169; 2004/0117129; 2004/0121309; 2004/0121310; 2004/0121311; 2004/0121312; 2004/0121313; 2004/0121314; 2004/0121315; 2004/0121329; 2004/0121335; 2004/0121340; 2004/0122598; 2004/0122857; 2004/0161770; 2004/0185438; 2004/0202997; 2004/0209260; 2004/0219517; 2004/0253583; 2004/0253619; 2005/0027459; 2005/0123952; 2005/0130196 2005/0142581; 2005/0164215; 2005/0266397; 2005/0270191; 2006/0014154; 2006/0121520; 2006/0205040; 2006/0240412; 2006/0259249; 2006/0275749; 2006/0275788; 2007/0087336; 2007/0087337; 2007/0087338 2007/0087339; 2007/0087340; 2007/0087341; 2007/0184434; 2007/0218467; 2007/0218467; 2007/0218489; 2007/0224614; 2007/0238116; 2007/0243544; 2007/0248969; WO2002/070664; WO2003/001976; WO2003/100035; WO2004/009849; WO2004/052175; WO2004/053076; WO2004/053141; WO2004/053164; WO2004/060278; WO2004/093644; WO 2004/101809; WO2004/111187; WO2005/023083; WO2005/023986; WO2005/024046; WO2005/033271; WO2005/036369; WO2005/086634; WO2005/089128; WO2005/091971; WO2005/092059; WO2005/094421; WO2005/098047; WO2005/116263; WO2005/117270; WO2006/019784; WO2006/034294; WO2006/071241; WO2006/094238; WO2006/116127; WO2006/135400; WO2007/014045; WO2007/047778; WO2007/086904; WO2007/100397; WO2007/118222; Ecker et al., Ibis T5000: a universal biosensor approach for microbiology. Nat Rev Microbiol. Jun. 3, 2008.; Ecker et al., The Microbial Rosetta Stone Database: A compilation of global and emerging infectious microorganisms and bioterrorist threat agents. BMC Microbiology. 2005. 5(1): 19.; Ecker et al., The Ibis T5000 Universal Biosensor: An Automated Platform for Pathogen Identification and Strain Typing. JALA. 2006. 6(11): 341-351.; Ecker et al., The Microbial Rosetta Stone Database: A common structure for microbial biosecurity threat agents. J Forensic Sci. 2005. 50(6): 1380-5.; Ecker et al., Identification of Acinetobacter species and genotyping of Acinetobacter baumannii by multilocus PCR and mass spectrometry. J Clin Microbiol. 2006 August; 44(8):2921-32.; Ecker et al., Rapid identification and strain-typing of respiratory pathogens for epidemic surveillance. Proc Natl Acad Sci USA. 2005 May 31; 102(22):8012-7. Epub 2005 May 23.; Wortmann et al., Genotypic Evolution of Acinetobacter baumannii Strains in an Outbreak Associated With War Trauma. Infect Control Hosp Epidemiol. 2008 June; 29(6):553-555.; Hannis et al., High-resolution genotyping of Campylobacter species by use of PCR and high-throughput mass spectrometry. J Clin Microbiol. 2008 April; 46(4):1220-5.; Blyn et al., Rapid detection and molecular serotyping of adenovirus by use of PCR followed by electrospray ionization mass spectrometry. J Clin Microbiol. 2008 February; 46(2):644-51.; Eshoo et al., Direct broad-range detection of alphaviruses in mosquito extracts. Virology. 2007 Nov. 25; 368(2):286-95.; Sampath et al., Global surveillance of emerging Influenza virus genotypes by mass spectrometry. PLoS ONE. 2007 May 30; 2(5):e489.; Sampath et al., Rapid identification of emerging infectious agents using PCR and electrospray ionization mass spectrometry. Ann N Y Acad. Sci. 2007 April; 1102:109-20.; Hujer et al., Analysis of antibiotic resistance genes in multidrug-resistant Acinetobacter sp. isolates from military and civilian patients treated at the Walter Reed Army Medical Center. Antimicrob Agents Chemother. 2006 December; 50(12):4114-23.; Hall et al., Base composition analysis of human mitochondrial DNA using electrospray ionization mass spectrometry: a novel tool for the identification and differentiation of humans. Anal Biochem. 2005 Sep. 1; 344(1):53-69.; Sampath et al., Rapid identification of emerging pathogens: coronavirus. Emerg Infect Dis. 2005 March; 11(3):373-9.; Jiang Y, Hofstadler S A. A highly efficient and automated method of purifying and desalting PCR products for analysis by electrospray ionization mass spectrometry. Anal Biochem. 2003. 316: 50-57.; Jiang et al., Mitochondrial DNA mutation detection by electrospray mass spectrometry. Clin Chem. 2006. 53(2): 195-203. Epub Dec 7.; Russell et al., Transmission dynamics and prospective environmental sampling of adenovirus in a military recruit setting. J Infect Dis. 2006. 194(7): 877-85. Epub Aug. 25, 2006.; Hofstadler et al., Detection of microbial agents using broad-range PCR with detection by mass spectrometry: The TIGER concept. Chapter in Encyclopedia of Rapid Microbiological Methods. 2006.; Hofstadler et al., Selective ion filtering by digital thresholding: A method to unwind complex ESI-mass spectra and eliminate signals from low molecular weight chemical noise. Anal Chem. 2006. 78(2): 372-378.; Hofstadler et al., TIGER: The Universal Biosensor. Int J Mass Spectrom. 2005. 242(1): 23-41.; Van Ert et al., Mass spectrometry provides accurate characterization of two genetic marker types in Bacillus anthracis. Biotechniques. 2004. 37(4): 642-4, 646, 648.; Sampath et al., Forum on Microbial Threats: Learning from SARS: Preparing for the Next Disease Outbreak—Workshop Summary. (ed. Knobler S E, Mahmoud A, Lemon S.) The National Academies Press, Washington, D.C. 2004.181-185.

In certain embodiments, bioagent identifying amplicons amenable to molecular mass determination produced by the primers described herein are either of a length, size or mass compatible with a particular mode of molecular mass determination, or compatible with a means of providing a fragmentation pattern in order to obtain fragments of a length compatible with a particular mode of molecular mass determination. Such means of providing a fragmentation pattern of an amplicon include, but are not limited to, cleavage with restriction enzymes or cleavage primers, sonication or other means of fragmentation. Thus, in some embodiments, bioagent identifying amplicons are larger than 200 nucleobases and are amenable to molecular mass determination following restriction digestion. Methods of using restriction enzymes and cleavage primers are well known to those with ordinary skill in the art.

In some embodiments, amplicons corresponding to bioagent identifying amplicons are obtained using the polymerase chain reaction (PCR). Other amplification methods may be used such as ligase chain reaction (LCR), low-stringency single primer PCR, and multiple strand displacement amplification (MDA). (Michael, S F., Biotechniques. 1994, 16:411-412 and Dean et al., Proc Natl Acad Sci USA. 2002, 99, 5261-5266).

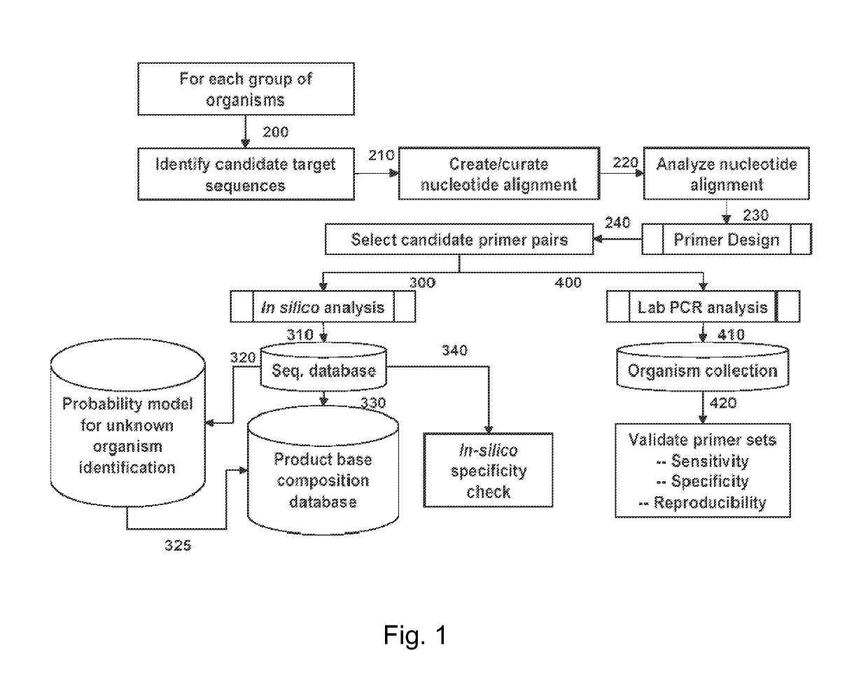

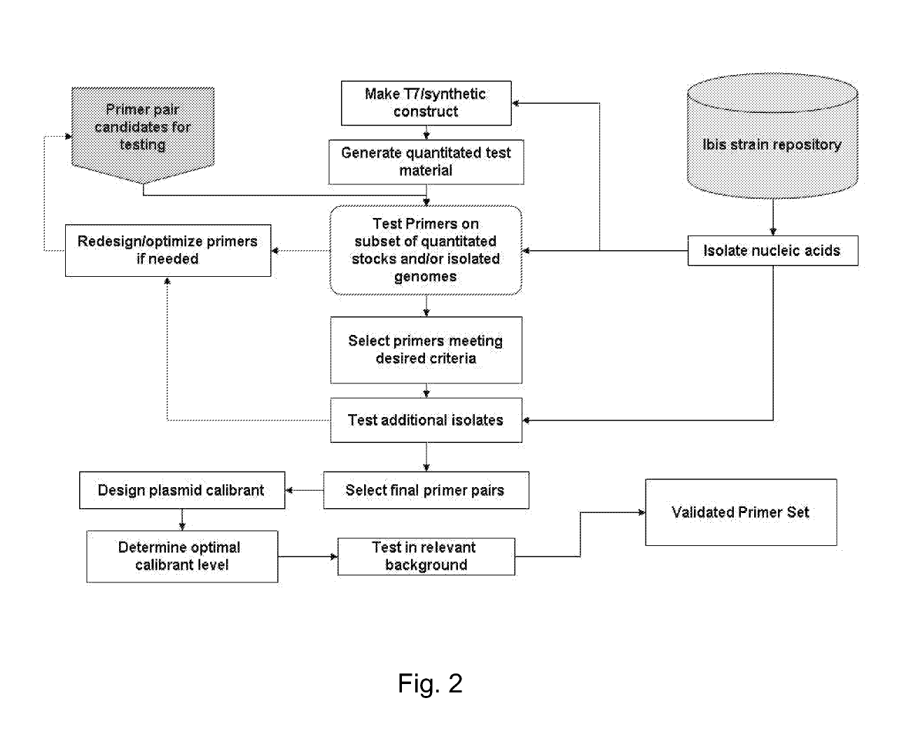

One embodiment of a process flow diagram used for primer selection and validation process is depicted in FIGS. 1 and 2. For each group of organisms, candidate target sequences are identified (200) from which nucleotide sequence alignments are created (210) and analyzed (220). Primers are then configured by selecting priming regions (230) to facilitate the selection of candidate primer pairs (240). The primer pair sequence is typically a “best fit” amongst the aligned sequences, such that the primer pair sequence may or may not be fully complementary to the hybridization region on any one of the bioagents in the alignment. Thus, best fit primer pair sequences are those with sufficient complementarity with two or more bioagents to hybridize with the two or more bioagents and generate an amplicon. The primer pairs are then subjected to in silico analysis by electronic PCR (ePCR) (300) wherein bioagent identifying amplicons are obtained from sequence databases such as GenBank or other sequence collections (310) and tested for specificity in silico (320). Bioagent identifying amplicons obtained from ePCR of GenBank sequences (310) may also be analyzed by a probability model which predicts the capability of a given amplicon to identify unknown bioagents. Preferably, the base compositions of amplicons with favorable probability scores are then stored in a base composition database (325). Alternatively, base compositions of the bioagent identifying amplicons obtained from the primers and GenBank sequences are directly entered into the base composition database (330). Candidate primer pairs (240) are validated by in vitro amplification by a method such as PCR analysis (400) of nucleic acid from a collection of organisms (410). Amplicons thus obtained are analyzed to confirm the sensitivity, specificity and reproducibility of the primers used to obtain the amplicons (420).

Synthesis of primers is well known and routine in the art. The primers may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed.

The primers typically are employed as compositions for use in methods for identification of bioagents as follows: a primer pair composition is contacted with nucleic acid (such as, for example, DNA) of an unknown species suspected of comprising Streptococcus pneumoniae. The nucleic acid is then amplified by a nucleic acid amplification technique, such as PCR for example, to obtain an amplicon that represents a bioagent identifying amplicon. The molecular mass of the strands of the double-stranded amplicon is determined by a molecular mass measurement technique such as mass spectrometry, for example. Preferably the two strands of the double-stranded amplicon are separated during the ionization process; however, they may be separated prior to mass spectrometry measurement. In some embodiments, the mass spectrometer is electrospray Fourier transform ion cyclotron resonance mass spectrometry (ESI-FTICR-MS), or electrospray time of flight mass spectrometry (ESI-TOF-MS). A list of possible base compositions may be generated for the molecular mass value obtained for each strand, and the choice of the base composition from the list is facilitated by matching the base composition of one strand with a complementary base composition of the other strand. A measured molecular mass or base composition calculated therefrom is then compared with a database of molecular masses or base compositions indexed to primer pairs and to known bioagents. A match between the measured molecular mass or base composition of the amplicon and the database molecular mass or base composition for that indexed primer pair correlates the measured molecular mass or base composition with an indexed bioagent, thus identifying the unknown bioagent (e.g., antibiotic resistant Streptococcus pneumoniae). In some embodiments, the primer pair used is at least one of the primer pairs of Tables 1 and 2. In some embodiments, the method is repeated using a different primer pair to resolve possible ambiguities in the identification process or to improve the confidence level for the identification assignment (triangulation identification). In some embodiments, for example, where the unknown is a novel, previously uncharacterized organism, the molecular mass or base composition from an amplicon generated from the unknown is matched with one or more best match molecular masses or base compositions from a database to predict a family, genus, species, sub-type, etc. of the unknown. Such information may assist further characterization of the unknown or provide a physician treating a patient infected by the unknown with a therapeutic agent best calculated to treat the patient.

In certain embodiments, Streptococcus pneumoniae is detected with the systems and methods of the present invention in combination with other bioagents, including viruses, bacteria, fungi, or other bioagents. In particular embodiments, a panel is employed that includes detection and identification of Streptococcus pneumoniae and other related or un-related bioagents. Such panels may be specific for a particular type of bioagent, or specific for a specific type of test (e.g., for testing the safety of blood, one may include commonly present viral pathogens such as HCV, HIV, and bacteria that can be contracted via a blood transfusion).

In some embodiments, a bioagent identifying amplicon may be produced using only a single primer (either the forward or reverse primer of any given primer pair), provided an appropriate amplification method is chosen, such as, for example, low stringency single primer PCR (LSSP-PCR).

In some embodiments, the oligonucleotide primers are broad range survey primers which hybridize to conserved regions of nucleic acid. The broad range primer may identify the unknown bioagent depending on which bioagent is in the sample. In other cases, the molecular mass or base composition of an amplicon does not provide sufficient resolution to identify the unknown bioagent as any one bioagent at or below the species level. These cases generally benefit from further analysis of one or more amplicons generated from at least one additional broad range survey primer pair, or from at least one additional division-wide primer pair, or from at least one additional drill-down primer pair. Identification of sub-species characteristics may be required, for example, to determine a clinical treatment of patient, or in rapidly responding to an outbreak of a new species, sub-type, etc. of pathogen to prevent an epidemic or pandemic.

One with ordinary skill in the art of design of amplification primers will recognize that a given primer need not hybridize with 100% complementarity in order to effectively prime the synthesis of a complementary nucleic acid strand in an amplification reaction. Primer pair sequences may be a “best fit” amongst the aligned bioagent sequences, thus they need not be fully complementary to the hybridization region of any one of the bioagents in the alignment. Moreover, a primer may hybridize over one or more segments such that intervening or adjacent segments are not involved in the hybridization event (e.g., a loop structure or a hairpin structure). The primers may comprise at least 70%, at least 75%, at least 80%, at least 85%, at least 90%, at least 95% or at least 99% sequence identity with any of the primers listed in Tables 1 and 2. Thus, in some embodiments, an extent of variation of 70% to 100%, or any range falling within, of the sequence identity is possible relative to the specific primer sequences disclosed herein. To illustrate, determination of sequence identity is described in the following example: a primer 20 nucleobases in length which is identical to another 20 nucleobase primer having two non-identical residues has 18 of 20 identical residues (18/20=0.9 or 90% sequence identity). In another example, a primer 15 nucleobases in length having all residues identical to a 15 nucleobase segment of primer 20 nucleobases in length would have 15/20=0.75 or 75% sequence identity with the 20 nucleobase primer. Percent identity need not be a whole number, for example when a 28 consecutive nucleobase primer is completely identical to a 31 consecutive nucleobase primer (28/31=0.9032 or 90.3% identical).

Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). In some embodiments, complementarity of primers with respect to the conserved priming regions of viral nucleic acid is between about 70% and about 80%. In other embodiments, homology, sequence identity or complementarity is between about 80% and about 90%. In yet other embodiments, homology, sequence identity or complementarity is at least 90%, at least 92%, at least 94%, at least 95%, at least 96%, at least 97%, at least 98%, at least 99% or is 100%.

In some embodiments, the primers described herein comprise at least 70%, at least 75%, at least 80%, at least 85%, at least 90%, at least 92%, at least 94%, at least 95%, at least 96%, at least 98%, or at least 99%, or 100% (or any range falling within) sequence identity with the primer sequences specifically disclosed herein.

In some embodiments, the oligonucleotide primers are 13 to 35 nucleobases in length (13 to 35 linked nucleotide residues). These embodiments comprise oligonucleotide primers 13, 14, 15, 16, 17, 18, 19, 20, 21, 22, 23, 24, 25, 26, 27, 28, 29, 30, 31, 32, 33, 34 or 35 nucleobases in length, or any range therewithin.

In some embodiments, any given primer comprises a modification comprising the addition of a non-templated T residue to the 5′ end of the primer (i.e., the added T residue does not necessarily hybridize to the nucleic acid being amplified). The addition of a non-templated T residue has an effect of minimizing the addition of non-templated A residues as a result of the non-specific enzyme activity of, e.g., Taq DNA polymerase (Magnuson et al., Biotechniques., 1996: 21, 700-709.), an occurrence which may lead to ambiguous results arising from molecular mass analysis.

Primers may contain one or more universal bases. Because any variation (i.e., due to codon wobble in the third position) in the conserved regions among species is likely to occur in the third position of a DNA (or RNA) triplet, oligonucleotide primers can be designed such that the nucleotide corresponding to this position is a base which can bind to more than one nucleotide, referred to herein as a “universal nucleobase.” For example, under this “wobble” base pairing, inosine (I) binds to U, C or A; guanine (G) binds to U or C, and uridine (U) binds to U or C. Other examples of universal nucleobases include nitroindoles such as 5-nitroindole or 3-nitropyrrole (Loakes et al., Nucleosides and Nucleotides., 1995, 14, 1001-1003.), the degenerate nucleotides dP or dK, an acyclic nucleoside analog containing 5-nitroindazole (Van Aerschot et al., Nucleosides and Nucleotides., 1995, 14, 1053-1056.) or the purine analog 1-(2-deoxy-beta-D-ribofuranosyl)-imidazole-4-carboxamide (Sala et al., Nucl. Acids Res., 1996, 24, 3302-3306.).

In some embodiments, to compensate for weaker binding by the wobble base, oligonucleotide primers are configured such that the first and second positions of each triplet are occupied by nucleotide analogs which bind with greater affinity than the unmodified nucleotide. Examples of these analogs include, but are not limited to, 2,6-diaminopurine which binds to thymine, 5-propynyluracil which binds to adenine and 5-propynylcytosine and phenoxazines, including G-clamp, which binds to G. Propynylated pyrimidines are described in U.S. Pat. Nos. 5,645,985, 5,830,653 and 5,484,908, each of which is commonly owned and is incorporated herein by reference in its entirety. Propynylated primers are described in U.S Pre-Grant Publication No. 2003-0170682; also commonly owned and incorporated herein by reference in its entirety. Phenoxazines are described in U.S. Pat. Nos. 5,502,177, 5,763,588, and 6,005,096, each of which is incorporated herein by reference in its entirety. G-clamps are described in U.S. Pat. Nos. 6,007,992 and 6,028,183, each of which is incorporated herein by reference in its entirety.

In some embodiments, non-template primer tags are used to increase the melting temperature (Tₘ) of a primer-template duplex in order to improve amplification efficiency. A non-template tag is at least three consecutive A or T nucleotide residues on a primer which are not complementary to the template. In any given non-template tag, A can be replaced by C or G, and T can also be replaced by C or G. Although Watson-Crick hybridization is not expected to occur for a non-template tag relative to the template, the extra hydrogen bond in a G-C pair relative to an A-T pair confers increased stability of the primer-template duplex and improves amplification efficiency for subsequent cycles of amplification when the primers hybridize to strands synthesized in previous cycles.

In other embodiments, propynylated tags may be used in a manner similar to that of the non-template tag, wherein two or more 5-propynylcytidine or 5-propynyluridine residues replace template matching residues on a primer. In other embodiments, a primer contains a modified internucleoside linkage such as a phosphorothioate linkage, for example.

In some embodiments, the primers contain mass-modifying tags. Reducing the total number of possible base compositions of a nucleic acid of specific molecular weight provides a means of avoiding a possible source of ambiguity in the determination of base composition of amplicons. Addition of mass-modifying tags to certain nucleobases of a given primer will result in simplification of de novo determination of base composition of a given bioagent identifying amplicon from its molecular mass.

In some embodiments, the mass modified nucleobase comprises one or more of the following: for example, 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine-5′-triphosphate, 5-iodo-2′-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate, )6-methyl-2′-deoxyguanosine-5′-triphosphate, N2-methyl-2′-deoxyguanosine-5′-triphosphate, 8-oxo-2′-deoxyguanosine-5′-triphosphate or thiothymidine-5′-triphosphate. In some embodiments, the mass-modified nucleobase comprises ¹⁵N or ¹³C, or both ¹³N and ¹³C.

In some embodiments, the molecular mass of a given bioagent (e.g., Streptococcus pneumoniae) identifying amplicon is determined by mass spectrometry. Mass spectrometry is intrinsically a parallel detection scheme without the need for radioactive or fluorescent labels, because an amplicon is identified by its molecular mass. The current state of the art in mass spectrometry is such that less than femtomole quantities of material can be analyzed to provide information about the molecular contents of the sample. An accurate assessment of the molecular mass of the material can be quickly obtained, irrespective of whether the molecular weight of the sample is several hundred, or in excess of one hundred thousand atomic mass units (amu) or Daltons.

In some embodiments, intact molecular ions are generated from amplicons using one of a variety of ionization techniques to convert the sample to the gas phase. These ionization methods include, but are not limited to, electrospray ionization (ESI), matrix-assisted laser desorption ionization (MALDI) and fast atom bombardment (FAB). Upon ionization, several peaks are observed from one sample due to the formation of ions with different charges. Averaging the multiple readings of molecular mass obtained from a single mass spectrum affords an estimate of molecular mass of the bioagent identifying amplicon. Electrospray ionization mass spectrometry (ESI-MS) is particularly useful for very high molecular weight polymers such as proteins and nucleic acids having molecular weights greater than 10 kDa, since it yields a distribution of multiply-charged molecules of the sample without causing a significant amount of fragmentation.

The mass detectors used include, but are not limited to, Fourier transform ion cyclotron resonance mass spectrometry (FT-ICR-MS), time of flight (TOF), ion trap, quadrupole, magnetic sector, Q-TOF, and triple quadrupole.

In some embodiments, assignment of previously unobserved base compositions (also known as “true unknown base compositions”) to a given phylogeny can be accomplished via the use of pattern classifier model algorithms. Base compositions, like sequences, may vary slightly from strain to strain, or serotype to serotype, within species, for example. In some embodiments, the pattern classifier model is the mutational probability model. In other embodiments, the pattern classifier is the polytope model. A polytope model is the mutational probability model that incorporates both the restrictions among strains and position dependence of a given nucleobase within a triplet. In certain embodiments, a polytope pattern classifier is used to classify a test or unknown organism according to its amplicon base composition.

In some embodiments, it is possible to manage this diversity by building “base composition probability clouds” around the composition constraints for each species. A “pseudo four-dimensional plot” may be used to visualize the concept of base composition probability clouds. Optimal primer design typically involves an optimal choice of bioagent identifying amplicons and maximizes the separation between the base composition signatures of individual bioagents. Areas where clouds overlap generally indicate regions that may result in a misclassification, a problem which is overcome by a triangulation identification process using bioagent identifying amplicons not affected by overlap of base composition probability clouds.

In some embodiments, base composition probability clouds provide the means for screening potential primer pairs in order to avoid potential misclassifications of base compositions. In other embodiments, base composition probability clouds provide the means for predicting the identity of an unknown bioagent whose assigned base composition has not been previously observed and/or indexed in a bioagent identifying amplicon base composition database due to evolutionary transitions in its nucleic acid sequence. Thus, in contrast to probe-based techniques, mass spectrometry determination of base composition does not require prior knowledge of the composition or sequence in order to make the measurement.

Provided herein is bioagent classifying information at a level sufficient to identify a given bioagent. Furthermore, the process of determining a previously unknown base composition for a given bioagent (for example, in a case where sequence information is unavailable) has utility by providing additional bioagent indexing information with which to populate base composition databases. The process of future bioagent identification is thus improved as additional base composition signature indexes become available in base composition databases.

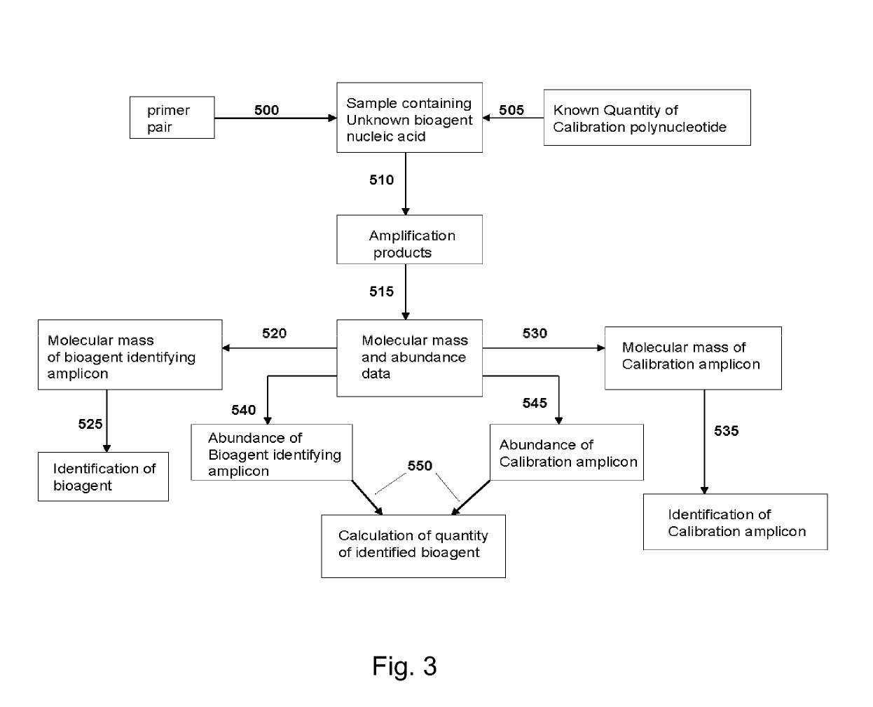

In some embodiments, the identity and quantity of an unknown bioagent may be determined using the process illustrated in FIG. 3. Primers (500) and a known quantity of a calibration polynucleotide (505) are added to a sample containing nucleic acid of an unknown bioagent. The total nucleic acid in the sample is then subjected to an amplification reaction (510) to obtain amplicons. The molecular masses of amplicons are determined (515) from which are obtained molecular mass and abundance data. The molecular mass of the bioagent identifying amplicon (520) provides for its identification (525) and the molecular mass of the calibration amplicon obtained from the calibration polynucleotide (530) provides for its quantification (535). The abundance data of the bioagent identifying amplicon is recorded (540) and the abundance data for the calibration data is recorded (545), both of which are used in a calculation (550) which determines the quantity of unknown bioagent in the sample.

In certain embodiments, a sample comprising an unknown bioagent is contacted with a primer pair which amplifies the nucleic acid from the bioagent, and a known quantity of a polynucleotide that comprises a calibration sequence. The amplification reaction then produces two amplicons: a bioagent identifying amplicon and a calibration amplicon. The bioagent identifying amplicon and the calibration amplicon are distinguishable by molecular mass while being amplified at essentially the same rate. Effecting differential molecular masses can be accomplished by choosing as a calibration sequence, a representative bioagent identifying amplicon (from a specific species of bioagent) and performing, for example, a 2-8 nucleobase deletion or insertion within the variable region between the two priming sites. The amplified sample containing the bioagent identifying amplicon and the calibration amplicon is then subjected to molecular mass analysis by mass spectrometry, for example. The resulting molecular mass analysis of the nucleic acid of the bioagent and of the calibration sequence provides molecular mass data and abundance data for the nucleic acid of the bioagent and of the calibration sequence. The molecular mass data obtained for the nucleic acid of the bioagent enables identification of the unknown bioagent by base composition analysis. The abundance data enables calculation of the quantity of the bioagent, based on the knowledge of the quantity of calibration polynucleotide contacted with the sample.

In some embodiments, construction of a standard curve in which the amount of calibration or calibrant polynucleotide spiked into the sample is varied provides additional resolution and improved confidence for the determination of the quantity of bioagent in the sample. Alternatively, the calibration polynucleotide can be amplified in its own reaction vessel or vessels under the same conditions as the bioagent. A standard curve may be prepared there from, and the relative abundance of the bioagent determined by methods such as linear regression. In some embodiments, multiplex amplification is performed where multiple bioagent identifying amplicons are amplified with multiple primer pairs which also amplify the corresponding standard calibration sequences. In this or other embodiments, the standard calibration sequences are optionally included within a single construct (preferably a vector) which functions as the calibration polynucleotide.

In some embodiments, the calibrant polynucleotide is used as an internal positive control to confirm that amplification conditions and subsequent analysis steps are successful in producing a measurable amplicon. Even in the absence of copies of the genome of a bioagent, the calibration polynucleotide gives rise to a calibration amplicon. Failure to produce a measurable calibration amplicon indicates a failure of amplification or subsequent analysis step such as amplicon purification or molecular mass determination. Reaching a conclusion that such failures have occurred is, in itself, a useful event. In some embodiments, the calibration sequence is comprised of DNA. In some embodiments, the calibration sequence is comprised of RNA.

In some embodiments, a calibration sequence is inserted into a vector which then functions as the calibration polynucleotide. In some embodiments, more than one calibration sequence is inserted into the vector that functions as the calibration polynucleotide. Such a calibration polynucleotide is herein termed a “combination calibration polynucleotide.” It should be recognized that the calibration method should not be limited to the embodiments described herein. The calibration method can be applied for determination of the quantity of any bioagent identifying amplicon when an appropriate standard calibrant polynucleotide sequence is designed and used.

In certain embodiments, primer pairs are configured to produce bioagent identifying amplicons within more conserved regions of a Streptococcus pneumoniae, while others produce bioagent identifying amplicons within regions that are may evolve more quickly. Primer pairs that characterize amplicons in a conserved region with low probability that the region will evolve past the point of primer recognition are useful, e.g., as a broad range survey-type primer. Primer pairs that characterize an amplicon corresponding to an evolving genomic region are useful, e.g., for distinguishing emerging bioagent strain variants.

The primer pairs described herein provide reagents, e.g., for identifying diseases caused by emerging species or strains or serotypes of Streptococcus pneumoniae (e.g., antibiotic resistant Streptococcus pneumoniae). Base composition analysis eliminates the need for prior knowledge of bioagent sequence to generate hybridization probes. Thus, in another embodiment, there is provided a method for determining the etiology of a particular stain or serotype when the process of identification of is carried out in a clinical setting, and even when a new strain or serotype is involved. This is possible because the methods may not be confounded by naturally occurring evolutionary variations.

Another embodiment provides a means of tracking the spread of any species or strain or serotype of Streptococcus pneumoniae when a plurality of samples obtained from different geographical locations are analyzed by methods described above in an epidemiological setting. For example, a plurality of samples from a plurality of different locations may be analyzed with primers which produce bioagent identifying amplicons, a subset of which identifies a specific strain or serotype. The corresponding locations of the members of the strain-containing or serotype-containing subset indicate the spread of the specific strain or serotype to the corresponding locations.

Also provided are kits for carrying out the methods described herein. In some embodiments, the kit may comprise a sufficient quantity of one or more primer pairs to perform an amplification reaction on a target polynucleotide from a bioagent to form a bioagent identifying amplicon. In some embodiments, the kit may comprise from one to one hundred primer pairs, from one to fifty primer pairs, one to twenty primer pairs, from one to ten primer pairs, from one to eight pairs, from one to five primer pairs, from one to three primer pairs, or from one to two primer pairs. In some embodiments, the kit may comprise one or more primer pairs recited in Table 1 or in Table 2. In certain embodiments, kits include all of the primer pairs recited in Table 1, or in Table 2, or in Table 1 and Table 2.

In some embodiments, the kit may also comprise a sufficient quantity of reverse transcriptase, a DNA polymerase, suitable nucleoside triphosphates (i.e., including any of those described above), a DNA ligase, and/or reaction buffer, or any combination thereof, for the amplification processes described above. A kit may further include instructions pertinent for the particular embodiment of the kit, such instructions describing the primer pairs and amplification conditions for operation of the method. In some embodiments, the kit further comprises instructions for analysis, interpretation and dissemination of data acquired by the kit. In other embodiments, instructions for the operation, analysis, interpretation and dissemination of the data of the kit are provided on computer readable media. A kit may also comprise amplification reaction containers such as microcentrifuge tubes, microtiter plates, and the like. A kit may also comprise reagents or other materials for isolating bioagent nucleic acid or bioagent identifying amplicons from amplification reactions, including, for example, detergents, solvents, or ion exchange resins which may be linked to magnetic beads. A kit may also comprise a table of measured or calculated molecular masses and/or base compositions of bioagents using the primer pairs of the kit.

The invention also provides systems that can be used to perform various assays relating to Streptococcus pneumoniae detection or identification. In certain embodiments, systems include mass spectrometers configured to detect molecular masses of amplicons produced using purified oligonucleotide primer pairs described herein. Other detectors that are optionally adapted for use in the systems of the invention are described further below. In some embodiments, systems also include controllers operably connected to mass spectrometers and/or other system components. In some of these embodiments, controllers are configured to correlate the molecular masses of the amplicons with bioagents to effect detection or identification. In some embodiments, controllers are configured to determine base compositions of the amplicons from the molecular masses of the amplicons. As described herein, the base compositions generally correspond to the Streptococcus pneumoniae serotype identities. In certain embodiments, controllers include, or are operably connected to, databases of known molecular masses and/or known base compositions of amplicons of known serotypes of Streptococcus pneumoniae (e.g., antibiotic resistant Streptococcus pneumoniae), and/or Streptococcus pneumoniae produced with the primer pairs described herein. Controllers are described further below.

In some embodiments, systems include one or more of the primer pairs described herein (e.g., in Table 1 and Table 2). In certain embodiments, the oligonucleotides are arrayed on solid supports, whereas in others, they are provided in one or more containers, e.g., for assays performed in solution. In certain embodiments, the systems also include at least one detector or detection component (e.g., a spectrometer) that is configured to detect detectable signals produced in the container or on the support. In addition, the systems also optionally include at least one thermal modulator (e.g., a thermal cycling device) operably connected to the containers or solid supports to modulate temperature in the containers or on the solid supports, and/or at least one fluid transfer component (e.g., an automated pipettor) that transfers fluid to and/or from the containers or solid supports, e.g., for performing one or more assays (e.g., nucleic acid amplification, real-time amplicon detection, etc.) in the containers or on the solid supports.

Detectors are typically structured to detect detectable signals produced, e.g., in or proximal to another component of the given assay system (e.g., in a container and/or on a solid support). Suitable signal detectors that are optionally utilized, or adapted for use, herein detect, e.g., fluorescence, phosphorescence, radioactivity, absorbance, refractive index, luminescence, or mass. Detectors optionally monitor one or a plurality of signals from upstream and/or downstream of the performance of, e.g., a given assay step. For example, detectors optionally monitor a plurality of optical signals, which correspond in position to “real-time” results. Exemplary detectors or sensors include photomultiplier tubes, CCD arrays, optical sensors, temperature sensors, pressure sensors, pH sensors, conductivity sensors, or scanning detectors. Detectors are also described in, e.g., Skoog et al., Principles of Instrumental Analysis, 5^{th} Ed., Harcourt Brace College Publishers (1998), Currell, Analytical Instrumentation: Performance Characteristics and Quality, John Wiley & Sons, Inc. (2000), Sharma et al., Introduction to Fluorescence Spectroscopy, John Wiley & Sons, Inc. (1999), Valeur, Molecular Fluorescence: Principles and Applications, John Wiley & Sons, Inc. (2002), and Gore, Spectrophotometry and Spectrofluorimetry: A Practical Approach, 2.sup.nd Ed., Oxford University Press (2000), which are each incorporated by reference herein in their entireties.

As mentioned above, the systems of the invention also typically include controllers that are operably connected to one or more components (e.g., detectors, databases, thermal modulators, fluid transfer components, robotic material handling devices, and the like) of the given system to control operation of the components. More specifically, controllers are generally included either as separate or integral system components that are utilized, e.g., to receive data from detectors (e.g., molecular masses, etc.), to effect and/or regulate temperature in the containers, or to effect and/or regulate fluid flow to or from selected containers. Controllers and/or other system components are optionally coupled to an appropriately programmed processor, computer, digital device, information appliance, or other logic device (e.g., including an analog to digital or digital to analog converter as needed), which functions to instruct the operation of these instruments in accordance with preprogrammed or user input instructions, receive data and information from these instruments, and interpret, manipulate and report this information to the user. Suitable controllers are generally known in the art and are available from various commercial sources.

Any controller or computer optionally includes a monitor, which is often a cathode ray tube (“CRT”) display, a flat panel display (e.g., active matrix liquid crystal display or liquid crystal display), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user. These components are illustrated further below.

The computer typically includes appropriate software for receiving user instructions, either in the form of user input into a set of parameter fields, e.g., in a graphic user interface (GUI), or in the form of preprogrammed instructions, e.g., preprogrammed for a variety of different specific operations. The software then converts these instructions to appropriate language for instructing the operation of one or more controllers to carry out the desired operation. The computer then receives the data from, e.g., sensors/detectors included within the system, and interprets the data, either provides it in a user understood format, or uses that data to initiate further controller instructions, in accordance with the programming.

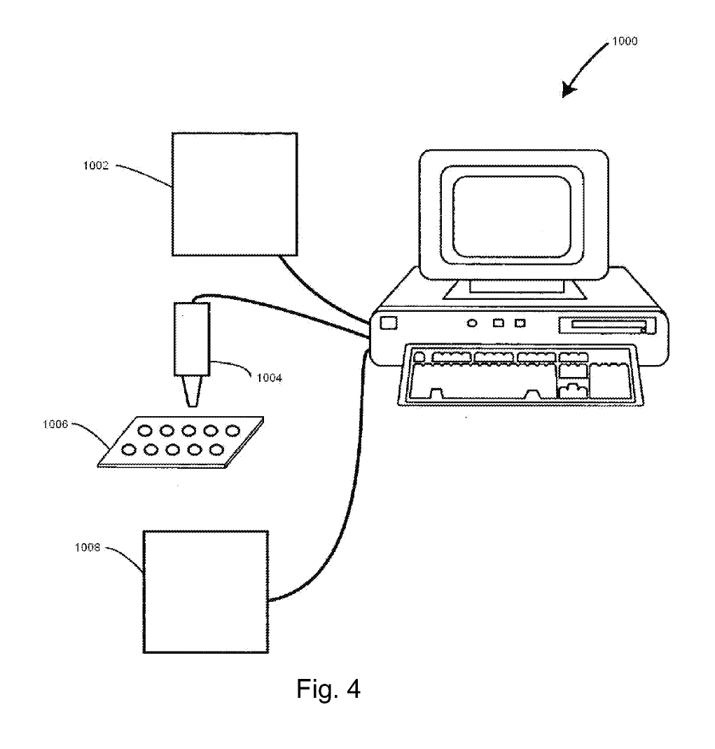

FIG. 4 is a schematic showing a representative system that includes a logic device in which various aspects of the present invention may be embodied. As will be understood by practitioners in the art from the teachings provided herein, aspects of the invention are optionally implemented in hardware and/or software. In some embodiments, different aspects of the invention are implemented in either client-side logic or server-side logic. As will be understood in the art, the invention or components thereof may be embodied in a media program component (e.g., a fixed media component) containing logic instructions and/or data that, when loaded into an appropriately configured computing device, cause that device to perform as desired. As will also be understood in the art, a fixed media containing logic instructions may be delivered to a viewer on a fixed media for physically loading into a viewer's computer or a fixed media containing logic instructions may reside on a remote server that a viewer accesses through a communication medium in order to download a program component.

More specifically, FIG. 4 schematically illustrates computer 1000 to which mass spectrometer 1002 (e.g., an ESI-TOF mass spectrometer, etc.), fluid transfer component 1004 (e.g., an automated mass spectrometer sample injection needle or the like), and database 1008 are operably connected. Optionally, one or more of these components are operably connected to computer 1000 via a server (not shown in FIG. 4). During operation, fluid transfer component 1004 typically transfers reaction mixtures or components thereof (e.g., aliquots comprising amplicons) from multi-well container 1006 to mass spectrometer 1002. Mass spectrometer 1002 then detects molecular masses of the amplicons. Computer 1000 then typically receives this molecular mass data, calculates base compositions from this data, and compares it with entries in database 1008 to identify species or strains of Streptococcus pneumoniae (e.g., antibiotic resistant Streptococcus pneumoniae) in a given sample. It will be apparent to one of skill in the art that one or more components of the system schematically depicted in FIG. 4 are optionally fabricated integral with one another (e.g., in the same housing).

While the present invention has been described with specificity in accordance with certain of its embodiments, the following examples serve only to illustrate the invention and are not intended to limit the same. In order that the invention disclosed herein may be more efficiently understood, examples are provided below. It should be understood that these examples are for illustrative purposes only and are not to be construed as limiting the invention in any manner.

### Example 1

### High-Throughput ESI-Mass Spectrometry Assay for Detection and Identification of Streptococcus pneumoniae

This example describes a Streptococcus pneumoniae (e.g., antibiotic resistant Streptococcus pneumoniae) pathogen identification assay which employs mass spectrometry determined base compositions for PCR amplicons derived from Streptococcus pneumoniae. The T5000 Biosensor System is a mass spectrometry based universal biosensor that uses mass measurements to derived base compositions of PCR amplicons to identify bioagents including, for example, bacteria, fungi, viruses and protozoa (S. A. Hofstadler et. al. Int. J. Mass Spectrom. (2005) 242:23-41, herein incorporated by reference in its entirety). For this Streptococcus pneumoniae assay primers from Table 1 and Table 2 may be employed to generate PCR amplicons. The base composition of the PCR amplicons can be determined and compared to a database of known Streptococcus pneumoniae (e.g., antibiotic resistant Streptococcus pneumoniae) base compositions to determine the identity of a Streptococcus pneumoniae in a sample. Table 1A shows exemplary primers pairs for detecting Streptococcus pneumoniae. Tables 1B to 1D provide additional information include hybridization coordinates of each primer and coordinates of reference amplicons with respect to reference sequences.

TABLE 1A
Primer Sequences

| Primer | | | SEQ |
|---|---|---|---|
| Pair | Primer | | ID |
| Number | Direction | Primer Sequence | NO |
| 3158 | Forward | TCACCGACTCAACTGCTGTACC | 3 |
| 3158 | Reverse | TGTTGAAGCCTGTGTTGCGTTGTA | 43 |
| 3160 | Forward | TCCACCTTTAAAGAAGATGGATTGGATGA | 6 |
| 3160 | Reverse | TCACACCCGACTCCACTGC | 46 |
| 3161 | Forward | TTGTATGAGGAATCCCTAAAAGCTATTAATGGAAT | 7 |
| 3161 | Reverse | TACTGTAGAGGGAATTCTGACACCTGC | 47 |
| 3162 | Forward | TGTACTAGCAGTTAGAGCGGCGAA | 8 |
| 3162 | Reverse | TCTATTATTCCTGAACTAGCTGCCTCTGAAT | 48 |
| 3163 | Forward | TAGAGTATGGCGTTGTAGCGGT | 9 |
| 3163 | Reverse | TCCTATTCCCGAATCTGCCAATATCTG | 49 |
| 3164 | Forward | TGGCGCATTGTGTATGCTACCAT | 11 |
| 3164 | Reverse | TGCCACGGAAGTTTAAATTGAAAGCC | 51 |
| 3165 | Forward | TTTGGTGATGCTGAATTAGCCTTTGG | 12 |
| 3165 | Reverse | TCCACCGTTCCATCCCAACC | 52 |
| 3166 | Forward | TCCTATTTGGGGATTAGGTATTTCAGACGG | 32 |
| 3166 | Reverse | TCCAACAGTTCTATCCATATGTTGTTCAATGG | 72 |
| 3167 | Forward | TACCTGAGATAAGCACTGTTCCTACGG | 14 |
| 3167 | Reverse | TCGTTCAGACAACCTATTTGCGTACTC | 54 |
| 3168 | Forward | TCCCAAGGAAATTTTCTAAAGAGTACAGC | 16 |
| 3168 | Reverse | TGCTAGTAACTCGTTGTTGACCGAA | 56 |
| 3169 | Forward | TGTACTCAGTCTTACTAGACGTAATGAACCC | 38 |
| 3169 | Reverse | TTGAAAGATAGCTAACAAACCAAAAATAGTCGT | 78 |
| 3170 | Forward | TCGTTCAACGACTAGGACGCTATTTGA | 17 |
| 3170 | Reverse | TTGCTGAATTGAGCCTCCTAGATAGGT | 57 |
| 3171 | Forward | TACAGCCGGGATTAAAGCGCC | 19 |
| 3171 | Reverse | TCTTGGGAAAGCGTATTTCTTTCATTCC | 59 |
| 3172 | Forward | TTTGTTTGGAAGAAGCTTATTAGGTTGGGA | 21 |
| 3172 | Reverse | TACTCCGTAACTGGTAGCTGATACGAA | 61 |
| 3173 | Forward | TAGACTTTTCTGCTATACATAGGTCAATGGC | 22 |
| 3173 | Reverse | TCCATTACCGAATAATATATTCAATATATTCCTACTCCA | 62 |
| 3174 | Forward | TGCGATTTTTGCTTTACCCTTTATGATGATG | 24 |
| 3174 | Reverse | TTTCCAACGAAACGTATCATCGCAAAATA | 64 |
| 3175 | Forward | TCATGAATCAAGCAGTGGCTATAAATCCTAA | 37 |
| 3175 | Reverse | TTTCAAGTTCTCCATCTCCAGCCAT | 77 |
| 3176 | Forward | TCCCGCTACTCTATAGAATGGAGTATATAAACTATGG | 26 |
| 3176 | Reverse | TCAAAGTTGCCAAAGCCAGCCA | 66 |
| 3177 | Forward | TTTCAAGGAAATCTAAGATATATCAATTGGTGGGA | 27 |
| 3177 | Reverse | TCACGCTTCAATTGTTCTATATCATGCTC | 67 |
| 3178 | Forward | TCGGTCGTGGAAGTTTCTCGC | 28 |
| 3178 | Reverse | TCCAATCCGACTAAGTCTTCAGTAAAAAACTTTAC | 68 |
| 3179 | Forward | TCCAGAGATTTTAGCTCTTAGTGCACTAAC | 29 |
| 3179 | Reverse | TAACAACTTTTGGAAGATACTGAACATAAAAAGTCAC | 69 |
| 3180 | Forward | TTTGCACCCTGACTTCACTAATGGGA | 31 |
| 3180 | Reverse | TGCTAAGCAATAAAATCCTTGGATTCCATTTGC | 71 |
| 3181 | Forward | TGCTAACGGTAAAGTGATAGCTAATAGTATGGA | 13 |
| 3181 | Reverse | TGAGATAGGATTGGTATACCGAATTCCCAT | 53 |
| 3182 | Forward | TGGGCAACCGATTTCTGGGC | 33 |
| 3182 | Reverse | TCCATTTTGCAGCTTCGTGCGA | 73 |
| 3183 | Forward | TCAATATTAGCCAAAAAGCACAGTATACCCC | 34 |
| 3183 | Reverse | TAAAAATACCATACATCCAAATGCTCTCTTATATG | 74 |
| 3257 | Forward | TGTGCCTTCTTTGTAGACAGCGATC | 20 |
| 3257 | Reverse | TGGAGCAAGTGGTTCTCCAAAGATAGA | 60 |
| 3258 | Forward | TTCGCAGAAGGCAAATTGCTTCA | 25 |
| 3258 | Reverse | TGTTGCTGAAGCGACTGTCTCAA | 65 |
| 3259 | Forward | TTAACCGCGACCGCTTTATTCTTTCA | 30 |
| 3259 | Reverse | TGACCTGGTGTTTTTGAACCCCATT | 70 |
| 3260 | Forward | TGATCCGACCCTAGCGGATGG | 5 |
| 3260 | Reverse | TATGGTGTCGCCAGGCATTCC | 45 |
| 3261 | Forward | TGAACATCACCATGAACGAAGGCATC | 35 |
| 3261 | Reverse | TCAAAACCTTGTCCTCTGGTGAGAGG | 75 |
| 3262 | Forward | TGCCTTCCGATATGACAGCCG | 10 |
| 3262 | Reverse | TGTAGTCATAAAAGGCAACGTCCTTGAC | 50 |
| 3263 | Forward | TGAAGTTGTCAAGGACGTTGCCTT | 15 |
| 3263 | Reverse | TGCACGGAAGGCTGTTTCTGC | 55 |
| 3364 | Forward | TGTGCTCCCGTCGATTCAAGAG | 2 |
| 3364 | Reverse | TGCGCTGGAAACAACAGACAAC | 42 |
| 3365 | Forward | TGCATTGCTAGAGATGGTTCCTTCAG | 4 |
| 3365 | Reverse | TCTTCCCATACTCTAGTGCAAACTTTGC | 44 |
| 3366 | Forward | TGACACTACTACAACATATGCAGCAGC | 1 |
| 3366 | Reverse | TAAGTTCGCAATCCAGCTTCAACATG | 41 |
| 3367 | Forward | TCGGATGGCGTCAGTCAGATTTC | 40 |
| 3367 | Reverse | TGCAGCTCAGAAGCATATTCTAAAGCA | 80 |
| 3387 | Forward | TTCCCAAAGCGTTCCGGTGT | 18 |
| 3387 | Reverse | TGATTAGTTGCTTGGTAAAATGCACCAG | 58 |
| 3388 | Forward | TGCAAGTGGGCACTGTGGA | 39 |
| 3388 | Reverse | TCTGCTCGTGACCGCATAAGG | 79 |
| 3389 | Forward | TCCTGGGAATTGGCACTCTTCTG | 23 |
| 3389 | Reverse | TCGGCAAATGTTGAAACCATACGC | 63 |
| 3522 | Forward | TGGAGTCTTGTCATGGAGTTATCGGTAT | 36 |
| 3522 | Reverse | TCAGCACTTCCAAGTCGTAATCTACC | 76 |

TABLE 1B
Primer Pair Names and Reference Amplicon Lengths

| Primer | | Reference |
|---|---|---|
| Pair | | Amplicon |
| Number | Primer Pair Name | Length |
| 3158 | CAP3C_Z47210-8662-9582_407_477 | 71 |
| 3160 | MNAB_CR931660-14353-15576_127_187 | 61 |
| 3161 | WZY_CR931662-7101-8273_823_900 | 78 |
| 3162 | WZY_AF316639-9499-10899_522_590 | 69 |
| 3163 | WZX_CR931648-11741-13165_116_189 | 74 |
| 3164 | WZY_CR931653-11178-12350_805_887 | 83 |
| 3165 | WZY_AF057294-8510-9703_844_932 | 89 |
| 3166 | WZY_AY163221-11-547_548_548 | 1 |
| 3167 | WCWH_CR931643-13317-14384_20_93 | 74 |
| 3168 | WZY_CR931668-11267-12529_469_541 | 73 |
| 3169 | WZY_CR931673-12088-13362_236_336 | 101 |
| 3170 | WZY_U09239-7573-8910_777_840 | 64 |
| 3171 | WCRH_CR931705-10365-11438_48_127 | 80 |
| 3172 | WZY_CR931664-7105-8277_420_480 | 61 |
| 3173 | WZY_CR931695-8928-10190_830_924 | 95 |
| 3174 | WZY_CR931710-13656-15086_483_569 | 87 |
| 3175 | WCIS_CR931644-7505-8569_584_652 | 69 |
| 3176 | WZY_Z83335-10238-11542_224_310 | 87 |
| 3177 | WCRG_CR931649-12120-13076_769_860 | 92 |
| 3178 | WZY_CR931703-7299-8669_380_461 | 82 |
| 3179 | WZY_CR931707-6889-8109_33_127 | 95 |
| 3180 | WZY_CR931663-7114-8313_423_501 | 79 |
| 3181 | WCIP_CR931670-10372-11355_405_474 | 70 |
| 3182 | WCIL_CR931679-9407-10522_421_466 | 46 |
| 3183 | WCWL_CR931642-8874-10064_349_422 | 74 |
| 3257 | SPNMLST-GDH_NC003098-1123551- | 141 |
| | 1124010_116_256 | |
| 3258 | SPNMLST-GKI_NC003098-600427- | 135 |
| | 600909_166_300 | |
| 3259 | SPNMLST-RECP_NC003098-1817785- | 139 |
| | 1817336_16_154 | |
| 3260 | SPNMLST-SPI_NC003098-364415- | 142 |
| | 363943_43_184 | |
| 3261 | SPNMLST-XPT_NC003098-1635367- | 117 |
| | 1635850_201_317 | |
| 3262 | SPNMLST-DDL_NC003098-1492971- | 140 |
| | 1492531_126_265 | |
| 3263 | SPNMLST-DDL_NC003098-1492971- | 139 |
| | 1492531_231_369 | |
| 3364 | WCWV_CR931682-10776-11900_831_912 | 82 |
| 3365 | WCIP_AF316640-8200-9186_510_619 | 110 |
| 3366 | CPS19AK_AF094575-11986-13077_288_374 | 87 |
| 3367 | SPNMLST-AROE_NC003098-1232155- | 137 |
| | 1231720_295_431 | |
| 3387 | WZX9N9L_CR931647-11738-13162_954_1049 | 96 |
| 3388 | WXY5_CR931637-6003-7208_544_632 | 89 |
| 3389 | WZY23A_CR931683-7578-8984_678_749 | 72 |
| 3522 | WZY13_CR931661-12410-13570_726_818_2 | 93 |

TABLE 1C
Individual Primer Pair Names Indicating Primer Hybridization Coordinates

| Primer | | |
|---|---|---|
| Pair | Primer | |
| Number | Direction | Individual Primer Name |
| 3158 | Forward | CAP3C_Z47210-8662-9582_407_428_F |
| 3158 | Reverse | CAP3C_Z47210-8662-9582_454_477_R |
| 3160 | Forward | MNAB_CR931660-14353-15576_127_155_F |
| 3160 | Reverse | MNAB_CR931660-14353-15576_169_187_R |
| 3161 | Forward | WZY_CR931662-7101-8273_823_857_F |
| 3161 | Reverse | WZY_CR931662-7101-8273_874_900_R |
| 3162 | Forward | WZY_AF316639-9499-10899_522_545_F |
| 3162 | Reverse | WZY_AF316639-9499-10899_560_590_R |
| 3163 | Forward | WZX_CR931648-11741-13165_116_137_F |
| 3163 | Reverse | WZX_CR931648-11741-13165_163_189_R |
| 3164 | Forward | WZY_CR931653-11178-12350_805_827_F |
| 3164 | Reverse | WZY_CR931653-11178-12350_862_887_R |
| 3165 | Forward | WZY_AF057294-8510-9703_844_869_F |
| 3165 | Reverse | WZY_AF057294-8510-9703_913_932_R |
| 3166 | Forward | WZY_AY163221-11-547_548_548_F |
| 3166 | Reverse | WZY_AY163221-11-547_548_548_R |
| 3167 | Forward | WCWH_CR931643-13317-14384_20_46_F |
| 3167 | Reverse | WCWH_CR931643-13317-14384_67_93_R |
| 3168 | Forward | WZY_CR931668-11267-12529_469_497_F |
| 3168 | Reverse | WZY_CR931668-11267-12529_517_541_R |
| 3169 | Forward | WZY_CR931673-12088-13362_236_266_F |
| 3169 | Reverse | WZY_CR931673-12088-13362_304_336_R |
| 3170 | Forward | WZY_U09239-7573-8910_777_803_F |
| 3170 | Reverse | WZY_U09239-7573-8910_814_840_R |
| 3171 | Forward | WCRH_CR931705-10365-11438_48_68_F |
| 3171 | Reverse | WCRH_CR931705-10365-11438_100_127_R |
| 3172 | Forward | WZY_CR931664-7105-8277_420_449_F |
| 3172 | Reverse | WZY_CR931664-7105-8277_454_480_R |
| 3173 | Forward | WZY_CR931695-8928-10190_830_860_F |
| 3173 | Reverse | WZY_CR931695-8928-10190_886_924_R |
| 3174 | Forward | WZY_CR931710-13656-15086_483_513_F |
| 3174 | Reverse | WZY_CR931710-13656-15086_541_569_R |
| 3175 | Forward | WCIS_CR931644-7505-8569_584_614_F |
| 3175 | Reverse | WCIS_CR931644-7505-8569_628_652_R |
| 3176 | Forward | WZY_Z83335-10238-11542_224_260_F |
| 3176 | Reverse | WZY_Z83335-10238-11542_289_310_R |
| 3177 | Forward | WCRG_CR931649-12120-13076_769_803_F |
| 3177 | Reverse | WCRG_CR931649-12120-13076_832_860_R |
| 3178 | Forward | WZY_CR931703-7299-8669_380_400_F |
| 3178 | Reverse | WZY_CR931703-7299-8669_427_461_R |
| 3179 | Forward | WZY_CR931707-6889-8109_33_62_F |
| 3179 | Reverse | WZY_CR931707-6889-8109_91_127_R |
| 3180 | Forward | WZY_CR931663-7114-8313_423_448_F |
| 3180 | Reverse | WZY_CR931663-7114-8313_469_501_R |
| 3181 | Forward | WCIP_CR931670-10372-11355_405_437_F |
| 3181 | Reverse | WCIP_CR931670-10372-11355_445_474_R |
| 3182 | Forward | WCIL_CR931679-9407-10522_421_440_F |
| 3182 | Reverse | WCIL_CR931679-9407-10522_445_466_R |
| 3183 | Forward | WCWL_CR931642-8874-10064_349_379_F |
| 3183 | Reverse | WCWL_CR931642-8874-10064_388_422_R |
| 3257 | Forward | SPNMLST-GDH_NC003098-1123551-1124010_116_140_F |
| 3257 | Reverse | SPNMLST-GDH_NC003098-1123551-1124010_230_256_R |
| 3258 | Forward | SPNMLST-GKI_NC003098-600427-600909_166_188_F |
| 3258 | Reverse | SPNMLST-GKI_NC003098-600427-600909_278_300_R |
| 3259 | Forward | SPNMLST-RECP_NC003098-1817785-1817336_16_41_F |
| 3259 | Reverse | SPNMLST-RECP_NC003098-1817785-1817336_130_154_R |
| 3260 | Forward | SPNMLST-SPI_NC003098-364415-363943_43_63_F |
| 3260 | Reverse | SPNMLST-SPI_NC003098-364415-363943_164_184_R |
| 3261 | Forward | SPNMLST-XPT_NC003098-1635367-1635850_201_226_F |
| 3261 | Reverse | SPNMLST-XPT_NC003098-1635367-1635850_292_317_R |
| 3262 | Forward | SPNMLST-DDL_NC003098-1492971-1492531_126_146_F |
| 3262 | Reverse | SPNMLST-DDL_NC003098-1492971-1492531_238_265_R |
| 3263 | Forward | SPNMLST-DDL_NC003098-1492971-1492531_231_254_F |
| 3263 | Reverse | SPNMLST-DDL_NC003098-1492971-1492531_349_369_R |
| 3364 | Forward | WCWV_CR931682-10776-11900_831_852_F |
| 3364 | Reverse | WCWV_CR931682-10776-11900_891_912_R |
| 3365 | Forward | WCIP_AF316640-8200-9186_510_535_F |
| 3365 | Reverse | WCIP_AF316640-8200-9186_592_619_R |
| 3366 | Forward | CPS19AK_AF094575-11986-13077_288_314_F |
| 3366 | Reverse | CPS19AK_AF094575-11986-13077_349_374_R |
| 3367 | Forward | SPNMLST-AROE_NC003098-1232155-1231720_295_317_F |
| 3367 | Reverse | SPNMLST-AROE_NC003098-1232155-1231720_405_431_R |
| 3387 | Forward | WZX9N9L_CR931647-11738-13162_954_973_F |
| 3387 | Reverse | WZX9N9L_CR931647-11738-13162_1022_1049_R |
| 3388 | Forward | WXY5_CR931637-6003-7208_544_562_F |
| 3388 | Reverse | WXY5_CR931637-6003-7208_612_632_R |
| 3389 | Forward | WZY23A_CR931683-7578-8984_678_700_F |
| 3389 | Reverse | WZY23A_CR931683-7578-8984_726_749_R |
| 3522 | Forward | WZY13_CR931661-12410-13570_726_753_F |
| 3522 | Reverse | WZY13_CR931661-12410-13570_793_818_R |

TABLE 1D
GenBank Accession Numbers and gi Numbers of Reference Sequences

| | Reference Sequence | |
|---|---|---|
| Primer Pair | GenBank Accession | Reference Sequence |
| Number | Number | GenBank gi Number |
| 3158 | Z47210 | 1658316 |
| 3160 | CR931660 | 68642944 |
| 3161 | CR931662 | 68642995 |
| 3162 | AF316639 | 13377403 |
| 3163 | CR931648 | 68642642 |
| 3164 | CR931653 | 68642762 |
| 3165 | AF057294 | 3818479 |
| 3166 | AY163221 | 37725550 |
| 3167 | CR931643 | 68642525 |
| 3168 | CR931668 | 68643161 |
| 3169 | CR931673 | 68643303 |
| 3170 | U09239 | 1881538 |
| 3171 | CR931705 | 68644178 |
| 3172 | CR931664 | 68643045 |
| 3173 | CR931695 | 68643918 |
| 3174 | CR931710 | 68644293 |
| 3175 | CR931644 | 68642552 |
| 3176 | Z83335 | 1944619 |
| 3177 | CR931649 | 68642666 |
| 3178 | CR931703 | 68644130 |
| 3179 | CR931707 | 68644228 |
| 3180 | CR931663 | 68643019 |
| 3181 | CR931670 | 68643219 |
| 3182 | CR931679 | 68643470 |
| 3183 | CR931642 | 68642497 |
| 3257 | NC_003098 | 15902044 |
| 3258 | NC_003098 | 15902044 |
| 3259 | NC_003098 | 15902044 |
| 3260 | NC_003098 | 15902044 |
| 3261 | NC_003098 | 15902044 |
| 3262 | NC_003098 | 15902044 |
| 3263 | NC_003098 | 15902044 |
| 3364 | CR931682 | 68643557 |
| 3365 | AF316640 | 13377419 |
| 3366 | AF094575 | 3907597 |
| 3367 | NC_003098 | 15902044 |
| 3387 | CR931647 | 68642621 |
| 3388 | CR931637 | 68642374 |
| 3389 | CR931683 | 68643586 |
| 3522 | CR931661 | 68642970 |

Multi-locus sequence typing (MLST) with detection and analysis of PCR products by electrospray ionization mass spectrometry (ESI-MS) is carried out using fragments of genes amplified by multiplex PCR from chromosomal DNA. (see, for example, Enright M C, Spratt B G. A multilocus sequence typing scheme for Streptococcus pneumoniae: identification of clones associated with serious invasive disease. Microbiology 1998; 144 (Pt 11):3049-60, incorporated by reference herein in its entirety.) PCR is performed in, for example, 40-μl reaction mixtures consisting of 10×PCR buffer, deoxynucleoside triphosphates (dNTPs), primers, genomic sample, and Taq polymerase (2.4 U per reaction mixture). The reactions are performed in 96-well plates (Bio-Rad, Hercules, Calif.) with an Eppendorf thermal cycler (Westbury N.Y.). The PCR reaction buffer consists of 4 U Amplitaq Gold (Applied Biosystems, Foster City, Calif. USA), 1× buffer II (Applied Biosystems, Foster City, Calif., USA), 2.0 mmol/L MgCl₂, 0.4 mol/L betaine and 800 μmol/L dNTP mix. PCR conditions used to amplify the sequences for PCR/electrospray ionization (ESI)-mass spectrometry analysis are: 95° C. for 10 min followed by 50 cycles of 95° C. for 30 s, 50° C. for 30 s, and 72° C. for 30 s.

After PCR amplification, 96-well plates containing amplicon mixtures are desalted using a protocol based on a weak anion-exchange method, and ESI-MS is then performed using the Ibis T5000 Biosensor System (Ibis Biosciences, Carlsbad, Calif.). Base compositions are derived using an algorithm constrained by Watson and Crick base pairing and acceptable mass error limits. Base composition signatures from multiple loci are used to generate the signature profile for each input sample. An automated algorithm computes the sequence types (STs) consistent with the PCR reactions performed on the input sample. The STs identified are then compared to STs of strains and serotypes in the MLST database to determine relationships to previously characterized strains (http:espneumoniae.mlst.net/) (see, for example, Feil E J, Li B C, Aanensen D M, Hanage W P, Spratt B G. eBURST: Inferring patterns of evolutionary descent among clusters of related bacterial genotypes from multilocus sequence typing data. J Bacteriol. 2004; 186:1518-30, incorporated by reference herein in its entirety.) The genotype profiles of the isolates are then compared with one another as well as with other isolates in the pneumococcal MLST database, using software available at the MLST website (http://www.mlst.net). Phylogenetic analysis of STs are performed using the program eBURST, that uses a model of bacterial evolution in which an ancestral genotype increases in frequency in a population, and diversifies to produce a cluster of closely-related genotypes descended from the founding genotype (http://eburst.mlst.net).

It is noted that the primer pairs in Table 1 could be combined into a single panel for detection of one or more Streptococcus pneumoniae (e.g., various serotypes of human Streptococcus pneumoniae). The primers and primer pairs of Table 1 could be used, for example, to detect human and animal infections. These primers and primer pairs may also be grouped (e.g., in panels or kits) for multiplex detection of other bioagents. In particular embodiments, the primers are used in assays for testing product safety. It is also noted that additional primer pairs could be contemplated for use with the panel for detection of Streptococcus pneumoniae including, but not limited to primer pairs specific for detection and identification of Streptococcus pneumoniae wciN, wchA and/or wciO gene regions.

Antibiotic resistance in a given strain or serotype of Streptococcus pneumoniae is indicated by bioagent identifying amplicons defined, for example, by primer pair SEQ ID NOS: 81:93, 82:94, 83:95, 84:96, 85:97, 86:98, 87:99, 88:100, 89:101, 90:102, 91:103, and 92:104 to determine the presence or absence of pbp2x, parC, gyrA, pbp2b, ermB, pbp1a, and mefE genoytpes (Table 2).

TABLE 2A
Primer Sequences

| Primer | | | |
|---|---|---|---|
| Pair | Primer | | |
| Number | Direction | Sequence | SEQ ID NO |
| 3523 | Forward | TGGCATTTAACGACGAAACTGGC | 87 |
| 3523 | Reverse | TCTGACGATAAGTTGAATAGATGACTGTCT | 99 |
| 3524 | Forward | TAGTGAGGACTTTGTTTGGCGTGAT | 81 |
| 3524 | Reverse | TCTCCACCTGGGAAGGTATTGTTATCAATAG | 93 |
| 3528 | Forward | TGCTGTGGAAGCTCTGGAGTATTC | 86 |
| 3528 | Reverse | TCTAAATTGCTGGTGCCAACAAACATATT | 98 |
| 3529 | Forward | TATCGTCTTCCAAGGTTCAGCTCC | 88 |
| 3529 | Reverse | TGACCATGTAGGCATTGGATGAATACTC | 100 |
| 3531 | Forward | TCGTATTACAGGGGATGTCATGGGTAAATA | 85 |
| 3531 | Reverse | TAACGGTAGCTCCACCATTGAGC | 97 |
| 3532 | Forward | TGTCGGGAACATCATGGGGAATTTC | 82 |
| 3532 | Reverse | TCTCACGATTCTTCCAGTTCTGTGAC | 94 |
| 3533 | Forward | TCCCTTCCTAAATGGTCTTGGAATCGACTA | 89 |
| 3533 | Reverse | TGAGCAGCAGCCATCTTTTCACTACTT | 101 |
| 3534 | Forward | TGGCTGGTAAAACAGGTACCTCTAACTA | 91 |
| 3534 | Reverse | TAGAGTAGCCTGTCCATACAGCCAT | 103 |
| 3535 | Forward | TACGCGTAAATATTCAATGGCTGTATGGAC | 92 |
| 3535 | Reverse | TGGTACGTCATCATAGAGCGGTAAACTTT | 104 |
| 4213 | Forward | TGAGTCATGCTGGAGCCAAAATTTAT | 83 |
| 4213 | Reverse | TGAAACAGGATTTCCCACTATTTCTTTTTG | 95 |
| 4214 | Forward | TTTATAACTGTTCCTGGGCAAAATGTAGC | 84 |
| 4214 | Reverse | TCGACCAGGTAACCTCCATTTTTCTC | 96 |
| 4266 | Forward | TCAGTATCATTAATCACTAGTGCCATCCTG | 90 |
| 4266 | Reverse | TCCTACTAATGAAGCCATAGACAAGACCAT | 102 |

TABLE 2B
Primer Pair Names and Reference Amplicon Lengths

| | | Reference |
|---|---|---|
| Primer | | Amplicon |
| Pair Number | Primer Pair Name | Length |
| 3523 | ERMB_DQ855649-1-941_405_490 | 86 |
| 3524 | PBP2X_AB119929-1-2253_954_1070 | 117 |
| 3528 | PBP2B_AB119906-1-2058_1320_1430 | 111 |
| 3529 | PBP2B_AB119906-1-2058_1251_1363 | 113 |
| 3531 | GYRA_DQ175173-1-2469_195_293 | 99 |
| 3532 | PARC_AF170996-1-2472_195_289 | 95 |
| 3533 | PBP1A_AB119773-1-2160_1338_1460 | 123 |
| 3534 | PBP1A_AB119773-1-2160_1661_1801 | 141 |
| 3535 | PBP1A_AB119773-1-2160_1761_1874 | 114 |
| 4213 | SPNEUMONIAPBP2X_AB119935-1- | 99 |
| | 2253_1414_1512 | |
| 4214 | SPNEUMONIAPBP2X-KSG_AB119935-1- | 86 |
| | 2253_1606_1691 | |
| 4266 | SPNEUMONIAEMEFE_AF274302-1125- | 105 |
| | 2342_55_159 | |

TABLE 2C
Individual Primer Pair Names Indicating Primer Hybridization Coordinates

| Primer | | |
|---|---|---|
| Pair | Primer | |
| Number | Direction | Individual Primer Name |
| 3523 | Forward | ERMB_DQ855649-1-941_405_490 |
| 3523 | Reverse | ERMB_DQ855649-1-941_461_490_R |
| 3524 | Forward | PBP2X_AB119929-1-2253_954_1070 |
| 3524 | Reverse | PBP2X_AB119929-1-2253_1040_1070_R |
| 3528 | Forward | PBP2B_AB119906-1-2058_1320_1430 |
| 3528 | Reverse | PBP2B_AB119906-1-2058_1402_1430_R |
| 3529 | Forward | PBP2B_AB119906-1-2058_1251_1363 |
| 3529 | Reverse | PBP2B_AB119906-1-2058_1336_1363_R |
| 3531 | Forward | GYRA_DQ175173-1-2469_195_293 |
| 3531 | Reverse | GYRA_DQ175173-1-2469_271_293_R |
| 3532 | Forward | PARC_AF170996-1-2472_195_289 |
| 3532 | Reverse | PARC_AF170996-1-2472_264_289_R |
| 3533 | Forward | PBP1A_AB119773-1-2160_1338_1460 |
| 3533 | Reverse | PBP1A_AB119773-1-2160_1434_1460_R |
| 3534 | Forward | PBP1A_AB119773-1-2160_1661_1801 |
| 3534 | Reverse | PBP1A_AB119773-1-2160_1777_1801_R |
| 3535 | Forward | PBP1A_AB119773-1-2160_1761_1874 |
| 3535 | Reverse | PBP1A_AB119773-1-2160_1846_1874_R |
| 4213 | Forward | SPNEUMONIAPBP2X_AB119935-1- |
| | | 2253_1414_1512 |
| 4213 | Reverse | SPNEUMONIAPBP2X_AB119935-1- |
| | | 2253_1483_1512_R |
| 4214 | Forward | SPNEUMONIAPBP2X-KSG_AB119935-1- |
| | | 2253_1606_1691 |
| 4214 | Reverse | SPNEUMONIAPBP2X-KSG_AB119935-1- |
| | | 2253_1666_1691_R |
| 4266 | Forward | SPNEUMONIAEMEFE_AF274302-1125- |
| | | 2342_55_159 |
| 4266 | Reverse | MEFE_AF274302-1125-2342_130_159_2_R |

TABLE 2D
GenBank Accession Numbers and gi Numbers of Reference Sequences

| | Reference | Reference |
|---|---|---|
| Primer Pair | Sequence GenBank | Sequence GenBank |
| Number | Accession Number | gi Number |
| 3523 | DQ855649 | 113196899 |
| 3524 | AB119929 | 38142228 |
| 3528 | AB119906 | 38142182 |
| 3529 | AB119906 | 38142182 |
| 3531 | DQ175173 | 73916211 |
| 3532 | AF170996 | 9230560 |
| 3533 | AB119773 | 38141916 |
| 3534 | AB119773 | 38141916 |
| 3535 | AB119773 | 38141916 |
| 4213 | AB119935 | 38142240 |
| 4214 | AB119935 | 38142240 |
| 4266 | AF274302 | 14578839 |

### Example 2

### De Novo Determination of Base Composition of Amplicons using Molecular Mass Modified Deoxynucleotide Triphosphates

Because the molecular masses of the four natural nucleobases fall within a narrow molecular mass range (A=313.058, G=329.052, C=289.046, T=304.046, values in Daltons—See, Table 5), a source of ambiguity in assignment of base composition may occur as follows: two nucleic acid strands having different base composition may have a difference of about 1 Da when the base composition difference between the two strands is G⇄A (−15.994) combined with C⇄T (+15.000). For example, one 99-mer nucleic acid strand having a base composition of A₂₇G₃₀C₂₁T₂₁ has a theoretical molecular mass of 30779.058, while another 99-mer nucleic acid strand having a base composition of A₂₆G₃₁C₂₂T₂₀ has a theoretical molecular mass of 30780.052 resulting in a molecular mass difference of only 0.994 Da. A 1 Da difference in molecular mass may be within the experimental error of a molecular mass measurement and thus, the relatively narrow molecular mass range of the four natural nucleobases imposes an uncertainty factor in this type of situation. One method for removing this theoretical 1 Da uncertainty factor uses amplification of a nucleic acid with one mass-tagged nucleobase and three natural nucleobases.

Addition of significant mass to one of the 4 nucleobases (dNTPs) in an amplification reaction, or in the primers themselves, will result in a significant difference in mass of the resulting amplicon (greater than 1 Da) arising from ambiguities such as the G⇄A combined with C⇄T event (Table 3). Thus, the same G⇄A (−15.994) event combined with 5-Iodo-C⇄T (−110.900) event would result in a molecular mass difference of 126.894 Da. The molecular mass of the base composition A₂₇G₃₀5-Indo-C₂₁T₂₁ (33422.958) compared with A₂₆G₃₁5Iodo-C₂₂T₂₀, (33549.852) provides a theoretical molecular mass difference is +126.894. The experimental error of a molecular mass measurement is not significant with regard to this molecular mass difference. Furthermore, the only base composition consistent with a measured molecular mass of the 99-mer nucleic acid is A₂₇G₃₀5-Iodo C₂₁T₂₁. In contrast, the analogous amplification without the mass tag has 18 possible base compositions.

TABLE 3
Molecular Masses of Natural Nucleobases and the Mass-Modified
Nucleobase 5-Iodo-C and Molecular Mass Differences Resulting from
Transitions

| Nucleobase | Molecular Mass | Transition | Δ Molecular Mass |
|---|---|---|---|
| A | 313.058 | A-->T | −9.012 |
| A | 313.058 | A-->C | −24.012 |
| A | 313.058 | A-->5-Iodo-C | 101.888 |
| A | 313.058 | A-->G | 15.994 |
| T | 304.046 | T-->A | 9.012 |
| T | 304.046 | T-->C | −15.000 |
| T | 304.046 | T-->5-Iodo-C | 110.900 |
| T | 304.046 | T-->G | 25.006 |
| C | 289.046 | C-->A | 24.012 |
| C | 289.046 | C-->T | 15.000 |
| C | 289.046 | C-->G | 40.006 |
| 5-Iodo-C | 414.946 | 5-Iodo-C-->A | −101.888 |
| 5-Iodo-C | 414.946 | 5-Iodo-C-->T | −110.900 |
| 5-Iodo-C | 414.946 | 5-Iodo-C-->G | −85.894 |
| G | 329.052 | G-->A | −15.994 |
| G | 329.052 | G-->T | −25.006 |
| G | 329.052 | G-->C | −40.006 |
| G | 329.052 | G-->5-Iodo-C | 85.894 |

Mass spectra of bioagent-identifying amplicons may be analyzed using a maximum-likelihood processor, as is widely used in radar signal processing. This processor first makes maximum likelihood estimates of the input to the mass spectrometer for each primer by running matched filters for each base composition aggregate on the input data. This includes the response to a calibrant for each primer.

The algorithm emphasizes performance predictions culminating in probability-of-detection versus probability-of-false-detection plots for conditions involving complex backgrounds of naturally occurring organisms and environmental contaminants. Matched filters consist of a priori expectations of signal values given the set of primers used for each of the bioagents. A genomic sequence database is used to define the mass base count matched filters. The database contains the sequences of known bioagents (e.g., Streptococcus pneumoniae) and includes threat organisms as well as benign background organisms. The latter is used to estimate and subtract the spectral signature produced by the background organisms. A maximum likelihood detection of known background organisms is implemented using matched filters and a running-sum estimate of the noise covariance. Background signal strengths are estimated and used along with the matched filters to form signatures which are then subtracted. The maximum likelihood process is applied to this “cleaned up” data in a similar manner employing matched filters for the organisms and a running-sum estimate of the noise-covariance for the cleaned up data.

The amplitudes of all base compositions of bioagent-identifying amplicons for each primer are calibrated and a final maximum likelihood amplitude estimate per organism is made based upon the multiple single primer estimates. Models of system noise are factored into this two-stage maximum likelihood calculation. The processor reports the number of molecules of each base composition contained in the spectra. The quantity of amplicon corresponding to the appropriate primer set is reported as well as the quantities of primers remaining upon completion of the amplification reaction.

Base count blurring may be carried out as follows. Electronic PCR can be conducted on nucleotide sequences of the desired bioagents to obtain the different expected base counts that could be obtained for each primer pair. See for example, Schuler, Genome Res., 1997; 7:541-50 (incorporated by reference herein in its entirety), or the e-PCR program available from National Center for Biotechnology Information (NCBI, NIH, Bethesda, Md.). In one embodiment, one or more spreadsheets from a workbook comprising a plurality of spreadsheets may be used (e.g., Microsoft Excel). First, in this example, there is a worksheet with a name similar to the workbook name; this worksheet contains the raw electronic PCR data. Second, there is a worksheet named “filtered bioagents base count” that contains bioagent name and base count; there is a separate record for each strain or serotype after removing sequences that are not identified with a genus and species, and removing all sequences for bioagents with less than 10 strains or serotypes. Third, there is a worksheet, “Sheet1” that contains the frequency of substitutions, insertions, or deletions for this primer pair. This data is generated by first creating a pivot table from the data in the “filtered bioagents base count” worksheet and then executing an Excel VBA macro. The macro creates a table of differences in base counts for bioagents of the same species, but different strains or serotypes.

Application of an exemplary script involves the user defining a threshold that specifies the fraction of the strains that are represented by the reference set of base counts for each bioagent. The reference set of base counts for each bioagent may contain as many different base counts as are needed to meet or exceed the threshold. The set of reference base counts is defined by selecting the most abundant strain's or serotypes base type composition and adding it to the reference set, and then the next most abundant strain's or serotypes base type composition is added until the threshold is met or exceeded.

For each base count not included in the reference base count set for the bioagent of interest, the script then proceeds to determine the manner in which the current base count differs from each of the base counts in the reference set. This difference may be represented as a combination of substitutions, Si=Xi, and insertions, Ii=Yi, or deletions, Di=Zi. If there is more than one reference base count, then the reported difference is chosen using rules to minimize the number of changes and, in instances with the same number of changes, to minimize the number of insertions or deletions. Therefore, the primary rule is to identify the difference with the minimum sum (Xi+Yi) or (Xi+Zi), e.g., one insertion rather than two substitutions. If there are two or more differences with the minimum sum, then the one that will be reported is the one that contains the most substitutions.

Differences between a base count and a reference composition are categorized as one, two, or more substitutions, one, two, or more insertions, one, two, or more deletions, and combinations of substitutions and insertions or deletions. The different classes of nucleobase changes and their probabilities of occurrence have been delineated in U.S. Patent Application Publication No. 2004209260 (U.S. application Ser. No. 10/418,514) which is incorporated herein by reference in entirety.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, internet web sites, and the like) cited in the present application is incorporated herein by reference in its entirety.

We claim:

1. A composition, comprising at least one purified oligonucleotide primer pair that comprises forward and reverse primers, wherein said primer pair comprises nucleic acid sequences that are substantially complementary to nucleic acid sequences of two or more different Streptococcus pneumoniae bioagents, wherein said primer pair is configured to produce amplicons comprising different base compositions that correspond to said two or more different bioagents.

2. The composition of claim 1, wherein said Streptococcus pneumoniae bioagents comprise antibiotic resistant Streptococcus pneumoniae bioagents.

3. The composition of claim 1, wherein said primer pair is configured to hybridize with conserved regions of said two or more different bioagents and flank variable regions of said two or more different bioagents.

4. The composition of claim 1, wherein said forward and reverse primers are about 15 to 35 nucleobases in length, and wherein the forward primer comprises at least 70%, sequence identity with a sequence selected from the group consisting of SEQ ID NOS: 1-40 and 81-92, and the reverse primer comprises at least 70% sequence identity with a sequence selected from the group consisting of SEQ ID NOS: 41-80 and 93-104.

5. The composition of claim 1, wherein said primer pair is selected from the group of primer pair sequences consisting of: SEQ ID NOS: 1:41, 2:42, 3:43, 4:44, 5:45, 6:46, 7:47, 8:48, 9:49, 10:50, 11:51, 12:52, 13:53, 14:54, 15:55, 16:56, 17:57, 18:58, 19:59, 20:60, 21:61, 22:62, 23:63, 24:64, 25:65, 26:66, 27:67, 28:68, 29:69, 30:70, 31:71, 32:72, 33:73, 34:74, 35:75, 36:76, 37:77, 38:78, 39:79, 40:80, 81:93, 82:94, 83:95, 84:96, 85:97, 86:98, 87:99, 88:100, 89:101, 90:102, 91:103, and 92:104.

6. The composition of claim 1, wherein said forward and reverse primers are about 15 to 35 nucleobases in length, and wherein:
the forward primer comprises at least 70%, sequence identity with the sequence of SEQ ID NO: 1, and the reverse primer comprises at least 70% sequence identity with the sequence of SEQ ID NO: 41;
the forward primer comprises at least 70% sequence identity with the sequence of SEQ ID NO: 2, and the reverse primer comprises at least 70% sequence identity with the sequence of SEQ ID NO: 42;
the forward primer comprises at least 70% sequence identity with the sequence of SEQ ID NO: 3, and the reverse primer comprises at least 70% sequence identity with the sequence of SEQ ID NO: 43;
the forward primer comprises at least 70% sequence identity with the sequence of SEQ ID NO: 4, and the reverse primer comprises at least 70% sequence identity with the sequence of SEQ ID NO: 44;
the forward primer comprises at least 70% sequence identity with the sequence of SEQ ID NO: 5, and the reverse primer comprises at least 70% sequence identity with the sequence of SEQ ID NO: 45;
the forward primer comprises at least 70% sequence identity with the sequence of SEQ ID NO: 6, and the reverse primer comprises at least 70% sequence identity with the sequence of SEQ ID NO: 46;
the forward primer comprises at least 70% sequence identity with the sequence of SEQ ID NO: 7, and the reverse primer comprises at least 70% sequence identity with the sequence of SEQ ID NO: 47;
the forward primer comprises at least 70% sequence identity with the sequence of SEQ ID NO: 8, and the reverse primer comprises at least 70% sequence identity with the sequence of SEQ ID NO: 48.
the forward primer comprises at least 70%, sequence identity with the sequence of SEQ ID NO: 9, and the reverse primer comprises at least 70% sequence identity with the sequence of SEQ ID NO: 49;
the forward primer comprises at least 70%, sequence identity with the sequence of SEQ ID NO: 10, and the reverse primer comprises at least 70% sequence identity with the sequence of SEQ ID NO: 50;
the forward primer comprises at least 70%, sequence identity with the sequence of SEQ ID NO: 11, and the reverse primer comprises at least 70% sequence identity with the sequence of SEQ ID NO: 51;
the forward primer comprises at least 70%, sequence identity with the sequence of SEQ ID NO: 12, and the reverse primer comprises at least 70% sequence identity with the sequence of SEQ ID NO: 52;
the forward primer comprises at least 70%, sequence identity with the sequence of SEQ ID NO: 13, and the reverse primer comprises at least 70% sequence identity with the sequence of SEQ ID NO: 53;
the forward primer comprises at least 70%, sequence identity with the sequence of SEQ ID NO: 14, and the reverse primer comprises at least 70% sequence identity with the sequence of SEQ ID NO: 54;
the forward primer comprises at least 70%, sequence identity with the sequence of SEQ ID NO: 15, and the reverse primer comprises at least 70% sequence identity with the sequence of SEQ ID NO: 55;
the forward primer comprises at least 70%, sequence identity with the sequence of SEQ ID NO: 16, and the reverse primer comprises at least 70% sequence identity with the sequence of SEQ ID NO: 56;
the forward primer comprises at least 70%, sequence identity with the sequence of SEQ ID NO: 17, and the reverse primer comprises at least 70% sequence identity with the sequence of SEQ ID NO: 57;
the forward primer comprises at least 70%, sequence identity with the sequence of SEQ ID NO: 18, and the reverse primer comprises at least 70% sequence identity with the sequence of SEQ ID NO: 58;
the forward primer comprises at least 70%, sequence identity with the sequence of SEQ ID NO: 19, and the reverse primer comprises at least 70% sequence identity with the sequence of SEQ ID NO: 59;
the forward primer comprises at least 70%, sequence identity with the sequence of SEQ ID NO: 20, and the reverse primer comprises at least 70% sequence identity with the sequence of SEQ ID NO: 60;
the forward primer comprises at least 70%, sequence identity with the sequence of SEQ ID NO: 21, and the reverse primer comprises at least 70% sequence identity with the sequence of SEQ ID NO: 61;
the forward primer comprises at least 70%, sequence identity with the sequence of SEQ ID NO: 22, and the reverse primer comprises at least 70% sequence identity with the sequence of SEQ ID NO: 62;
the forward primer comprises at least 70%, sequence identity with the sequence of SEQ ID NO: 23, and the reverse primer comprises at least 70% sequence identity with the sequence of SEQ ID NO: 63;
the forward primer comprises at least 70%, sequence identity with the sequence of SEQ ID NO: 24, and the reverse primer comprises at least 70% sequence identity with the sequence of SEQ ID NO: 64;
the forward primer comprises at least 70%, sequence identity with the sequence of SEQ ID NO: 25, and the reverse primer comprises at least 70% sequence identity with the sequence of SEQ ID NO: 65;
the forward primer comprises at least 70%, sequence identity with the sequence of SEQ ID NO: 26, and the reverse primer comprises at least 70% sequence identity with the sequence of SEQ ID NO: 66;
the forward primer comprises at least 70%, sequence identity with the sequence of SEQ ID NO: 27, and the reverse primer comprises at least 70% sequence identity with the sequence of SEQ ID NO: 67;
the forward primer comprises at least 70%, sequence identity with the sequence of SEQ ID NO: 28, and the reverse primer comprises at least 70% sequence identity with the sequence of SEQ ID NO: 68;
the forward primer comprises at least 70%, sequence identity with the sequence of SEQ ID NO: 29, and the reverse primer comprises at least 70% sequence identity with the sequence of SEQ ID NO: 69;
the forward primer comprises at least 70%, sequence identity with the sequence of SEQ ID NO: 30, and the reverse primer comprises at least 70% sequence identity with the sequence of SEQ ID NO: 70;
the forward primer comprises at least 70%, sequence identity with the sequence of SEQ ID NO: 31, and the reverse primer comprises at least 70% sequence identity with the sequence of SEQ ID NO: 71;
the forward primer comprises at least 70%, sequence identity with the sequence of SEQ ID NO: 32, and the reverse primer comprises at least 70% sequence identity with the sequence of SEQ ID NO: 72;
the forward primer comprises at least 70%, sequence identity with the sequence of SEQ ID NO: 33, and the reverse primer comprises at least 70% sequence identity with the sequence of SEQ ID NO: 73;
the forward primer comprises at least 70%, sequence identity with the sequence of SEQ ID NO: 34, and the reverse primer comprises at least 70% sequence identity with the sequence of SEQ ID NO: 74;
the forward primer comprises at least 70%, sequence identity with the sequence of SEQ ID NO: 35, and the reverse primer comprises at least 70% sequence identity with the sequence of SEQ ID NO: 75;
the forward primer comprises at least 70%, sequence identity with the sequence of SEQ ID NO: 36, and the reverse primer comprises at least 70% sequence identity with the sequence of SEQ ID NO: 76;
the forward primer comprises at least 70%, sequence identity with the sequence of SEQ ID NO: 37, and the reverse primer comprises at least 70% sequence identity with the sequence of SEQ ID NO: 77;
the forward primer comprises at least 70%, sequence identity with the sequence of SEQ ID NO: 38, and the reverse primer comprises at least 70% sequence identity with the sequence of SEQ ID NO: 78;
the forward primer comprises at least 70%, sequence identity with the sequence of SEQ ID NO: 39, and the reverse primer comprises at least 70% sequence identity with the sequence of SEQ ID NO: 79;
the forward primer comprises at least 70%, sequence identity with the sequence of SEQ ID NO: 40, and the reverse primer comprises at least 70% sequence identity with the sequence of SEQ ID NO: 80;
the forward primer comprises at least 70%, sequence identity with the sequence of SEQ ID NO: 81, and the reverse primer comprises at least 70% sequence identity with the sequence of SEQ ID NO: 93;
the forward primer comprises at least 70%, sequence identity with the sequence of SEQ ID NO: 82, and the reverse primer comprises at least 70% sequence identity with the sequence of SEQ ID NO: 94;
the forward primer comprises at least 70%, sequence identity with the sequence of SEQ ID NO: 83, and the reverse primer comprises at least 70% sequence identity with the sequence of SEQ ID NO: 95;
the forward primer comprises at least 70%, sequence identity with the sequence of SEQ ID NO: 84, and the reverse primer comprises at least 70% sequence identity with the sequence of SEQ ID NO: 96;
the forward primer comprises at least 70%, sequence identity with the sequence of SEQ ID NO: 85, and the reverse primer comprises at least 70% sequence identity with the sequence of SEQ ID NO: 97;
the forward primer comprises at least 70%, sequence identity with the sequence of SEQ ID NO: 86, and the reverse primer comprises at least 70% sequence identity with the sequence of SEQ ID NO: 98;
the forward primer comprises at least 70%, sequence identity with the sequence of SEQ ID NO: 87, and the reverse primer comprises at least 70% sequence identity with the sequence of SEQ ID NO: 99;
the forward primer comprises at least 70%, sequence identity with the sequence of SEQ ID NO: 88, and the reverse primer comprises at least 70% sequence identity with the sequence of SEQ ID NO: 100;
the forward primer comprises at least 70%, sequence identity with the sequence of SEQ ID NO: 89, and the reverse primer comprises at least 70% sequence identity with the sequence of SEQ ID NO: 101;
the forward primer comprises at least 70%, sequence identity with the sequence of SEQ ID NO: 90, and the reverse primer comprises at least 70% sequence identity with the sequence of SEQ ID NO: 102;
the forward primer comprises at least 70%, sequence identity with the sequence of SEQ ID NO: 91, and the reverse primer comprises at least 70% sequence identity with the sequence of SEQ ID NO: 103; and
the forward primer comprises at least 70%, sequence identity with the sequence of SEQ ID NO: 92, and the reverse primer comprises at least 70% sequence identity with the sequence of SEQ ID NO: 104.

7. The composition of claim 1, wherein said different base compositions identify said two or more different bioagents at genus levels, species levels, sub-species levels, strain levels, serogroup levels, serotype levels, serovar levels, or genotype levels.

8. The composition of claim 1, wherein said two or more amplicons are 45 to 200 nucleobases in length.

9. A kit comprising the composition of claim 1.

10. The composition of claim 1, wherein said primer pair amplifies a portion of a gene selected from the group consisting of: pbp2x, parC, gyrA, pbp2b, ermB, pbp1a, and mefE.

11. The composition of claim 1, wherein a non-templated T residue on the 5′-end of said forward and/or reverse primer is removed.

12. The composition of claim 1, wherein said forward and/or reverse primer further comprises a non-templated T residue on the 5′-end.

13. The composition of claim 1, wherein said forward and/or reverse primer comprises at least one molecular mass modifying tag.

14. The composition of claim 1, wherein said forward and/or reverse primer comprises at least one modified nucleobase.

15. The composition of claim 14, wherein said modified nucleobase is 5-propynyluracil or 5-propynylcytosine.

16. The composition of claim 14, wherein said modified nucleobase is a mass modified nucleobase.

17. The composition of claim 16, wherein said mass modified nucleobase is 5-Iodo-C.

18. The composition of claim 14, wherein said modified nucleobase is a universal nucleobase.

19. The composition of claim 18, wherein said universal nucleobase is inosine.

20. A composition comprising an isolated primer 15-35 bases in length selected from the group consisting of SEQ ID NOS: 1-104.

21. A kit, comprising at least one purified oligonucleotide primer pair that comprises forward and reverse primers that are about 20 to 35 nucleobases in length, and wherein said forward primer comprises at least 70% sequence identity with a sequence selected from the group consisting of SEQ ID NOS: 1-40 and 81-92, and said reverse primer comprises at least 70% sequence identity with a sequence selected from the group consisting of SEQ ID NOS: 41-80 and 93-104.

22. A method of determining the presence of Streptococcus pneumoniae in at least one sample, the method comprising:
(a) amplifying one or more segments of at least one nucleic acid from said sample using at least one purified oligonucleotide primer pair that comprises forward and reverse primers that are about 20 to 35 nucleobases in length, and wherein said forward primer comprises at least 70% sequence identity with a sequence selected from the group consisting of SEQ ID NOS: 1-40 and 81-92, and said reverse primer comprises at least 70% sequence identity with a sequence selected from the group consisting of SEQ ID NOS: 41-80 and 93-104 to produce at least one amplification product; and
(b) detecting said amplification product, thereby determining said presence of said Streptococcus pneumoniae in said sample.

23. The method of claim 22, wherein said Streptococcus pneumoniae is antibiotic resistant Streptococcus pneumoniae.

24. The method of claim 22, wherein (a) comprises amplifying said one or more segments of said at least one nucleic acid from at least two samples obtained from different geographical locations to produce at least two amplification products, and (b) comprises detecting said amplification products, thereby tracking an epidemic spread of said Streptococcus pneumoniae.

25. The method of claim 22, wherein (b) comprises determining an amount of said Streptococcus pneumoniae in said sample.

26. The method of claim 22, wherein (b) comprises detecting a molecular mass of said amplification product.

27. The method of claim 22, wherein (b) comprises determining a base composition of said amplification product, wherein said base composition identifies the number of A residues, C residues, T residues, G residues, U residues, analogs thereof and/or mass tag residues thereof in said amplification product, whereby said base composition indicates the presence of Streptococcus pneumoniae in said sample or identifies said Streptococcus pneumoniae in said sample.

28. The method of claim 27, comprising comparing said base composition of said amplification product to calculated or measured base compositions of amplification products of one or more known Streptococcus pneumoniae present in a database with the proviso that sequencing of said amplification product is not used to indicate the presence of or to identify said Streptococcus pneumoniae, wherein a match between said determined base composition and said calculated or measured base composition in said database indicates the presence of or identifies said Streptococcus pneumoniae.

29. A method of identifying one or more antibiotic resistant Streptococcus pneumoniae bioagents in a sample, the method comprising:
(a) amplifying two or more segments of a nucleic acid from said one or more antibiotic resistant Streptococcus pneumoniae bioagents in said sample with two or more oligonucleotide primer pairs to obtain two or more amplification products;
(b) determining two or more molecular masses and/or base compositions of said two or more amplification products; and
(c) comparing said two or more molecular masses and/or said base compositions of said two or more amplification products with known molecular masses and/or known base compositions of amplification products of known antibiotic resistant Streptococcus pneumoniae bioagents produced with said two or more primer pairs to identify said one or more antibiotic resistant Streptococcus pneumoniae bioagents in said sample.

30. The method of claim 29, comprising identifying said one or more antibiotic resistant Streptococcus pneumoniae bioagents in said sample using three, four, five, six, seven, eight or more primer pairs.

31. The method of claim 29, wherein said one or more antibiotic resistant Streptococcus pneumoniae bioagents in said sample cannot be identified using a single primer pair of said two or more primer pairs.

32. The method of claim 29, comprising obtaining said two or more molecular masses of said two or more amplification products via mass spectrometry.

33. The method of claim 29, comprising calculating said two or more base compositions from said two or more molecular masses of said two or more amplification products.

34. The method of claim 29, wherein said two or more primer pairs amplifies a portion of a gene selected from the group consisting of: pbp2x, parC, gyrA, pbp2b, ermB, pbp1a, and mefE.

35. The method of claim 29, wherein said two or more primer pairs comprise two or more purified oligonucleotide primer pairs that each comprise forward and reverse primers that are about 20 to 35 nucleobases in length, and wherein said forward primers comprise at least 70% sequence identity with a sequence selected from the group consisting of SEQ ID NOS: 1-40 and 81-92, and said reverse primers comprise at least 70% sequence identity with a sequence selected from the group consisting of SEQ ID NOS: 41-80 and 93-104 to obtain an amplification product.

36. The method of claim 29, wherein said primer pairs are selected from the group of primer pair sequences consisting of: SEQ ID NOS: 1:41, 2:42, 3:43, 4:44, 5:45, 6:46, 7:47, 8:48, 9:49, 10:50, 11:51, 12:52, 13:53, 14:54, 15:55, 16:56, 17:57, 18:58, 19:59, 20:60, 21:61, 22:62, 23:63, 24:64, 25:65, 26:66, 27:67, 28:68, 29:69, 30:70, 31:71, 32:72, 33:73, 34:74, 35:75, 36:76, 37:77, 38:78, 39:79, 40:80, 81:93, 82:94, 83:95, 84:96, 85:97, 86:98, 87:99, 88:100, 89:101, 90:102, 91:103, and 92:104.

37. The method of claim 29, wherein said determining said two or more molecular masses and/or base compositions is conducted without sequencing said two or more amplification products.

38. The method of claim 29, wherein said one or more antibiotic resistant Streptococcus pneumoniae bioagents in said sample cannot be identified using a single primer pair of said two or more primer pairs.

39. The method of claim 29, wherein said one or more antibiotic resistant Streptococcus pneumoniae bioagents in a sample are identified by comparing three or more molecular masses and/or base compositions of three or more amplification products with a database of known molecular masses and/or known base compositions of amplification products of known antibiotic resistant Streptococcus pneumoniae bioagents produced with said three or more primer pairs.

40. The method of claim 29, wherein said two or more segments of said nucleic acid are amplified from a single gene.

41. The method of claim 29, wherein said two or more segments of said nucleic acid are amplified from different genes.

42. The method of claim 29, wherein members of said primer pairs hybridize to conserved regions of said nucleic acid that flank a variable region.

43. The method of claim 42, wherein said variable region varies between at least two of said antibiotic resistant Streptococcus pneumoniae bioagents.

44. The method of claim 42, wherein said variable region uniquely varies between at least five of said antibiotic resistant Streptococcus pneumoniae bioagents.

45. The method of claim 29, wherein said two or more amplification products obtained in (a) comprise major classification and subgroup identifying amplification products.

46. The method of claim 45, comprising comparing said molecular masses and/or said base compositions of said two or more amplification products to calculated or measured molecular masses or base compositions of amplification products of known antibiotic resistant Streptococcus pneumoniae bioagents in a database comprising genus specific amplification products, species specific amplification products, strain specific amplification products, serovar specific amplification products, serogroup specific amplification products, serotype specific amplification products or nucleotide polymorphism specific amplification products produced with said two or more oligonucleotide primer pairs, wherein one or more matches between said two or more amplification products and one or more entries in said database identifies said one or more antibiotic resistant Streptococcus pneumoniae bioagents, classifies a major classification of said one or more antibiotic resistant Streptococcus pneumoniae bioagents, and/or differentiates between subgroups of known and unknown antibiotic resistant Streptococcus pneumoniae bioagents in said sample.

47. The method of claim 46, wherein said major classification of said one or more antibiotic resistant Streptococcus pneumoniae bioagents comprises a strain or genotype classification of said one or more antibiotic resistant Streptococcus pneumoniae bioagents.

48. The method of claim 46, wherein said subgroups of known and unknown antibiotic resistant Streptococcus pneumoniae bioagents comprise family, strain, serovar, serogroup, serotype and nucleotide variations of said one or more antibiotic resistant Streptococcus pneumoniae bioagents.

49. A system, comprising:
(a) a mass spectrometer configured to detect one or more molecular masses of amplicons produced using at least one purified oligonucleotide primer pair that comprises forward and reverse primers, wherein said primer pair comprises nucleic acid sequences that are substantially complementary to nucleic acid sequences of two or more different Streptococcus pneumoniae bioagents; and
(b) a controller operably connected to said mass spectrometer, said controller configured to correlate said molecular masses of said amplicons with one or more Streptococcus pneumoniae bioagent identities.

50. The system of claim 49, wherein said Streptococcus pneumoniae bioagent identities comprise antibiotic resistant Streptococcus pneumoniae bioagent identities.

51. The system of claim 49, wherein said Streptococcus pneumoniae bioagent identities are at genus, species, sub-species, strain, serovar, serogroup, serotype and/or genotype levels.

52. The system of claim 49, wherein said forward and reverse primers are about 15 to 35 nucleobases in length, and wherein the forward primer comprises at least 70%, at least 80%, at least 90%, at least 95%, or at least 100% sequence identity with a sequence selected from the group consisting of SEQ ID NOS: 1-40 and 81-92, and the reverse primer comprises at least 70% sequence identity with a sequence selected from the group consisting of SEQ ID NOS: 41-80 and 93-104.

53. The system of claim 49, wherein said primer pair is selected from the group of primer pair sequences consisting of: SEQ ID NOS: 1:41, 2:42, 3:43, 4:44, 5:45, 6:46, 7:47, 8:48, 9:49, 10:50, 11:51, 12:52, 13:53, 14:54, 15:55, 16:56, 17:57, 18:58, 19:59, 20:60, 21:61, 22:62, 23:63, 24:64, 25:65, 26:66, 27:67, 28:68, 29:69, 30:70, 31:71, 32:72, 33:73, 34:74, 35:75, 36:76, 37:77, 38:78, 39:79, 40:80, 81:93, 82:94, 83:95, 84:96, 85:97, 86:98, 87:99, 88:100, 89:101, 90:102, 91:103, and 92:104.

54. The system of claim 49, wherein said controller is configured to determine base compositions of said amplicons from said molecular masses of said amplicons, which base compositions correspond to said one or more Streptococcus pneumoniae bioagent identities.

55. The system of claim 49, wherein said controller comprises or is operably connected to a database of known molecular masses and/or known base compositions of amplicons of known Streptococcus pneumoniae bioagents produced with said primer pair.

56. A purified oligonucleotide primer pair, comprising a forward primer and a reverse primer that each independently comprises 14 to 40 consecutive nucleobases selected from the primer pair sequences shown in Table 1 and/or Table 2, which primer pair is configured to generate an amplicon between about 50 and 150 consecutive nucleobases in length.


SEQUENCE LISTING (104 sequences)

SEQ ID NO: 1 - DNA, 27 bp, Artificial Sequence
  Primer
tgacactactacaacatatgcagcagc

SEQ ID NO: 2 - DNA, 22 bp, Artificial Sequence
  Primer
tgtgctcccgtcgattcaagag

SEQ ID NO: 3 - DNA, 22 bp, Artificial Sequence
  Primer
tcaccgactcaactgctgtacc

SEQ ID NO: 4 - DNA, 26 bp, Artificial Sequence
  Primer
tgcattgctagagatggttccttcag

SEQ ID NO: 5 - DNA, 21 bp, Artificial Sequence
  Primer
tgatccgaccctagcggatgg

SEQ ID NO: 6 - DNA, 29 bp, Artificial Sequence
  Primer
tccacctttaaagaagatggattggatga

SEQ ID NO: 7 - DNA, 35 bp, Artificial Sequence
  Primer
ttgtatgaggaatccctaaaagctattaatggaat

SEQ ID NO: 8 - DNA, 24 bp, Artificial Sequence
  Primer
tgtactagcagttagagcggcgaa

SEQ ID NO: 9 - DNA, 22 bp, Artificial Sequence
  Primer
tagagtatggcgttgtagcggt

SEQ ID NO: 10 - DNA, 21 bp, Artificial Sequence
  Primer
tgccttccgatatgacagccg

SEQ ID NO: 11 - DNA, 23 bp, Artificial Sequence
  Primer
tggcgcattgtgtatgctaccat

SEQ ID NO: 12 - DNA, 26 bp, Artificial Sequence
  Primer
tttggtgatgctgaattagcctttgg

SEQ ID NO: 13 - DNA, 33 bp, Artificial Sequence
  Primer
tgctaacggtaaagtgatagctaatagtatgga

SEQ ID NO: 14 - DNA, 27 bp, Artificial Sequence
  Primer
tacctgagataagcactgttcctacgg

SEQ ID NO: 15 - DNA, 24 bp, Artificial Sequence
  Primer
tgaagttgtcaaggacgttgcctt

SEQ ID NO: 16 - DNA, 29 bp, Artificial Sequence
  Primer
tcccaaggaaattttctaaagagtacagc

SEQ ID NO: 17 - DNA, 27 bp, Artificial Sequence
  Primer
tcgttcaacgactaggacgctatttga

SEQ ID NO: 18 - DNA, 20 bp, Artificial Sequence
  Primer
ttcccaaagcgttccggtgt

SEQ ID NO: 19 - DNA, 21 bp, Artificial Sequence
  Primer
tacagccgggattaaagcgcc

SEQ ID NO: 20 - DNA, 25 bp, Artificial Sequence
  Primer
tgtgccttctttgtagacagcgatc

SEQ ID NO: 21 - DNA, 30 bp, Artificial Sequence
  Primer
tttgtttggaagaagcttattaggttggga

SEQ ID NO: 22 - DNA, 31 bp, Artificial Sequence
  Primer
tagacttttctgctatacataggtcaatggc

SEQ ID NO: 23 - DNA, 23 bp, Artificial Sequence
  Primer
tcctgggaattggcactcttctg

SEQ ID NO: 24 - DNA, 31 bp, Artificial Sequence
  Primer
tgcgatttttgctttaccctttatgatgatg

SEQ ID NO: 25 - DNA, 23 bp, Artificial Sequence
  Primer
ttcgcagaaggcaaattgcttca

SEQ ID NO: 26 - DNA, 37 bp, Artificial Sequence
  Primer
tcccgctactctatagaatggagtatataaactatgg

SEQ ID NO: 27 - DNA, 35 bp, Artificial Sequence
  Primer
tttcaaggaaatctaagatatatcaattggtggga

SEQ ID NO: 28 - DNA, 21 bp, Artificial Sequence
  Primer
tcggtcgtggaagtttctcgc

SEQ ID NO: 29 - DNA, 30 bp, Artificial Sequence
  Primer
tccagagattttagctcttagtgcactaac

SEQ ID NO: 30 - DNA, 26 bp, Artificial Sequence
  Primer
ttaaccgcgaccgctttattctttca

SEQ ID NO: 31 - DNA, 26 bp, Artificial Sequence
  Primer
tttgcaccctgacttcactaatggga

SEQ ID NO: 32 - DNA, 30 bp, Artificial Sequence
  Primer
tcctatttggggattaggtatttcagacgg

SEQ ID NO: 33 - DNA, 20 bp, Artificial Sequence
  Primer
tgggcaaccgatttctgggc

SEQ ID NO: 34 - DNA, 31 bp, Artificial Sequence
  Primer
tcaatattagccaaaaagcacagtatacccc

SEQ ID NO: 35 - DNA, 26 bp, Artificial Sequence
  Primer
tgaacatcaccatgaacgaaggcatc

SEQ ID NO: 36 - DNA, 28 bp, Artificial Sequence
  Primer
tggagtcttgtcatggagttatcggtat

SEQ ID NO: 37 - DNA, 31 bp, Artificial Sequence
  Primer
tcatgaatcaagcagtggctataaatcctaa

SEQ ID NO: 38 - DNA, 31 bp, Artificial Sequence
  Primer
tgtactcagtcttactagacgtaatgaaccc

SEQ ID NO: 39 - DNA, 19 bp, Artificial Sequence
  Primer
tgcaagtgggcactgtgga

SEQ ID NO: 40 - DNA, 23 bp, Artificial Sequence
  Primer
tcggatggcgtcagtcagatttc

SEQ ID NO: 41 - DNA, 26 bp, Artificial Sequence
  Primer
taagttcgcaatccagcttcaacatg

SEQ ID NO: 42 - DNA, 22 bp, Artificial Sequence
  Primer
tgcgctggaaacaacagacaac

SEQ ID NO: 43 - DNA, 24 bp, Artificial Sequence
  Primer
tgttgaagcctgtgttgcgttgta

SEQ ID NO: 44 - DNA, 28 bp, Artificial Sequence
  Primer
tcttcccatactctagtgcaaactttgc

SEQ ID NO: 45 - DNA, 21 bp, Artificial Sequence
  Primer
tatggtgtcgccaggcattcc

SEQ ID NO: 46 - DNA, 19 bp, Artificial Sequence
  Primer
tcacacccgactccactgc

SEQ ID NO: 47 - DNA, 27 bp, Artificial Sequence
  Primer
tactgtagagggaattctgacacctgc

SEQ ID NO: 48 - DNA, 31 bp, Artificial Sequence
  Primer
tctattattcctgaactagctgcctctgaat

SEQ ID NO: 49 - DNA, 27 bp, Artificial Sequence
  Primer
tcctattcccgaatctgccaatatctg

SEQ ID NO: 50 - DNA, 28 bp, Artificial Sequence
  Primer
tgtagtcataaaaggcaacgtccttgac

SEQ ID NO: 51 - DNA, 26 bp, Artificial Sequence
  Primer
tgccacggaagtttaaattgaaagcc

SEQ ID NO: 52 - DNA, 20 bp, Artificial Sequence
  Primer
tccaccgttccatcccaacc

SEQ ID NO: 53 - DNA, 30 bp, Artificial Sequence
  Primer
tgagataggattggtataccgaattcccat

SEQ ID NO: 54 - DNA, 27 bp, Artificial Sequence
  Primer
tcgttcagacaacctatttgcgtactc

SEQ ID NO: 55 - DNA, 21 bp, Artificial Sequence
  Primer
tgcacggaaggctgtttctgc

SEQ ID NO: 56 - DNA, 25 bp, Artificial Sequence
  Primer
tgctagtaactcgttgttgaccgaa

SEQ ID NO: 57 - DNA, 27 bp, Artificial Sequence
  Primer
ttgctgaattgagcctcctagataggt

SEQ ID NO: 58 - DNA, 28 bp, Artificial Sequence
  Primer
tgattagttgcttggtaaaatgcaccag

SEQ ID NO: 59 - DNA, 28 bp, Artificial Sequence
  Primer
tcttgggaaagcgtatttctttcattcc

SEQ ID NO: 60 - DNA, 27 bp, Artificial Sequence
  Primer
tggagcaagtggttctccaaagataga

SEQ ID NO: 61 - DNA, 27 bp, Artificial Sequence
  Primer
tactccgtaactggtagctgatacgaa

SEQ ID NO: 62 - DNA, 39 bp, Artificial Sequence
  Primer
tccattaccgaataatatattcaatatattcctactcca

SEQ ID NO: 63 - DNA, 24 bp, Artificial Sequence
  Primer
tcggcaaatgttgaaaccatacgc

SEQ ID NO: 64 - DNA, 29 bp, Artificial Sequence
  Primer
tttccaacgaaacgtatcatcgcaaaata

SEQ ID NO: 65 - DNA, 23 bp, Artificial Sequence
  Primer
tgttgctgaagcgactgtctcaa

SEQ ID NO: 66 - DNA, 22 bp, Artificial Sequence
  Primer
tcaaagttgccaaagccagcca

SEQ ID NO: 67 - DNA, 29 bp, Artificial Sequence
  Primer
tcacgcttcaattgttctatatcatgctc

SEQ ID NO: 68 - DNA, 35 bp, Artificial Sequence
  Primer
tccaatccgactaagtcttcagtaaaaaactttac

SEQ ID NO: 69 - DNA, 37 bp, Artificial Sequence
  Primer
taacaacttttggaagatactgaacataaaaagtcac

SEQ ID NO: 70 - DNA, 25 bp, Artificial Sequence
  Primer
tgacctggtgtttttgaaccccatt

SEQ ID NO: 71 - DNA, 33 bp, Artificial Sequence
  Primer
tgctaagcaataaaatccttggattccatttgc

SEQ ID NO: 72 - DNA, 32 bp, Artificial Sequence
  Primer
tccaacagttctatccatatgttgttcaatgg

SEQ ID NO: 73 - DNA, 22 bp, Artificial Sequence
  Primer
tccattttgcagcttcgtgcga

SEQ ID NO: 74 - DNA, 35 bp, Artificial Sequence
  Primer
taaaaataccatacatccaaatgctctcttatatg

SEQ ID NO: 75 - DNA, 26 bp, Artificial Sequence
  Primer
tcaaaaccttgtcctctggtgagagg

SEQ ID NO: 76 - DNA, 26 bp, Artificial Sequence
  Primer
tcagcacttccaagtcgtaatctacc

SEQ ID NO: 77 - DNA, 25 bp, Artificial Sequence
  Primer
tttcaagttctccatctccagccat

SEQ ID NO: 78 - DNA, 33 bp, Artificial Sequence
  Primer
ttgaaagatagctaacaaaccaaaaatagtcgt

SEQ ID NO: 79 - DNA, 21 bp, Artificial Sequence
  Primer
tctgctcgtgaccgcataagg

SEQ ID NO: 80 - DNA, 27 bp, Artificial Sequence
  Primer
tgcagctcagaagcatattctaaagca

SEQ ID NO: 81 - DNA, 25 bp, Artificial Sequence
  Primer
tagtgaggactttgtttggcgtgat

SEQ ID NO: 82 - DNA, 25 bp, Artificial Sequence
  Primer
tgtcgggaacatcatggggaatttc

SEQ ID NO: 83 - DNA, 26 bp, Artificial Sequence
  Primer
tgagtcatgctggagccaaaatttat

SEQ ID NO: 84 - DNA, 29 bp, Artificial Sequence
  Primer
tttataactgttcctgggcaaaatgtagc

SEQ ID NO: 85 - DNA, 30 bp, Artificial Sequence
  Primer
tcgtattacaggggatgtcatgggtaaata

SEQ ID NO: 86 - DNA, 24 bp, Artificial Sequence
  Primer
tgctgtggaagctctggagtattc

SEQ ID NO: 87 - DNA, 23 bp, Artificial Sequence
  Primer
tggcatttaacgacgaaactggc

SEQ ID NO: 88 - DNA, 24 bp, Artificial Sequence
  Primer
tatcgtcttccaaggttcagctcc

SEQ ID NO: 89 - DNA, 30 bp, Artificial Sequence
  Primer
tcccttcctaaatggtcttggaatcgacta

SEQ ID NO: 90 - DNA, 30 bp, Artificial Sequence
  Primer
tcagtatcattaatcactagtgccatcctg

SEQ ID NO: 91 - DNA, 28 bp, Artificial Sequence
  Primer
tggctggtaaaacaggtacctctaacta

SEQ ID NO: 92 - DNA, 30 bp, Artificial Sequence
  Primer
tacgcgtaaatattcaatggctgtatggac

SEQ ID NO: 93 - DNA, 31 bp, Artificial Sequence
  Primer
tctccacctgggaaggtattgttatcaatag

SEQ ID NO: 94 - DNA, 26 bp, Artificial Sequence
  Primer
tctcacgattcttccagttctgtgac

SEQ ID NO: 95 - DNA, 30 bp, Artificial Sequence
  Primer
tgaaacaggatttcccactatttctttttg

SEQ ID NO: 96 - DNA, 26 bp, Artificial Sequence
  Primer
tcgaccaggtaacctccatttttctc

SEQ ID NO: 97 - DNA, 23 bp, Artificial Sequence
  Primer
taacggtagctccaccattgagc

SEQ ID NO: 98 - DNA, 29 bp, Artificial Sequence
  Primer
tctaaattgctggtgccaacaaacatatt

SEQ ID NO: 99 - DNA, 30 bp, Artificial Sequence
  Primer
tctgacgataagttgaatagatgactgtct

SEQ ID NO: 100 - DNA, 28 bp, Artificial Sequence
  Primer
tgaccatgtaggcattggatgaatactc

SEQ ID NO: 101 - DNA, 27 bp, Artificial Sequence
  Primer
tgagcagcagccatcttttcactactt

SEQ ID NO: 102 - DNA, 30 bp, Artificial Sequence
  Primer
tcctactaatgaagccatagacaagaccat

SEQ ID NO: 103 - DNA, 25 bp, Artificial Sequence
  Primer
tagagtagcctgtccatacagccat

SEQ ID NO: 104 - DNA, 29 bp, Artificial Sequence
  Primer
tggtacgtcatcatagagcggtaaacttt